SEQUENCE LISTING (44 sequences)

SEQ ID NO: 1 - DNA, 7003 bp, Artificial
  misc_difference (1)..(7003): Plasmid
ggcatccgcttacagacaagctgtgaccgtctccgggagctgcatgtgtcagagtttcaccgtcatcaccgaaacgcgcgagacgaaagggcctcgtgatacgctatttttataggttaatgtcatgataataatggttcttagacgtcaggtggcacttttcggggaaatgtgcgcggaacccgtatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggtggagggggccgtaggagtagcccgcaccattcaacgatcaaccgatggccgttatgtggcggtgtcagcccggggtaatttttattccacctgggcaccgggacaaacggaatggactccccataaccgcaattcttcccgacgcttacagaccatgggctatggcaaggacggtcaactatggctgttggcccgggggggacaactccagttcagcaccgatcccgatgcagaggaatggagcgatgtgattgctccccaggataaaggtagttggggtctgctcgatctgtctttccgtacccctgaagaagtatgggtagcgggggccagcggtaacctcttgatgagtcaagacggggggcaaacctgggccaaggacactggggtagaagatattccagccaatctttatcgggtggtgttcctcagtccggaaaaaggatttgtgttggggcaggatgggattttgctcaaatataaccccagcaccgaggtggcaatggttccctaggcggctcacaaaatagtagactagactctacttgctttgcatttgtcagtcaatgttgttttgaaaaattgaaggagaacacaaaatgtcagggactaccggcgagcgtccattttccgatattgtcaccagcattcgctactgggtgatccacagcatcaccatcccgatgttgtttattgctggttggttgtttgtcagcacgggcttagcctacgatgcttttggcactccccgccccgatgaatatttcacccagacccgtcaagagttgcccattctccaggaacgctacgacattaatcaggaaattcaagagtttaatcaataaaacatttaattgttcttttttagttggtaattaacaatggcaacccaaaatcctaatcaaccggttacttatcccatttttacggtgcgctggctggcggttcacaccctggcggtgccctctgtcttctttgtcggggcgatcgccgcgatgcaatttattcaacgctaggagtttttcatggacagaaattcaaacccaaaccgccaaccggtggaattgaaccgcacttctttatacctgggtctattgttggtggctgtgttggggattttgttctccagctatttctttaactaaacttttttaatacgcaatttaggaggcatggtatgttcgcagaaggcagaatccctttgtgggtggtgggtgtagtggccggtattggcgccattggtgttctagggttatttttctacggagcctatgctggtttaggttcttccatgtaatcgagggctagagtcgacctgcaggcatgcaagcttatcgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgatgataagctgtcaaacatgagaattcttgaagacgaaagggcctcttgcgggatatcatccattccgacagcatcgccagtcactatggcgtgctgctagccgccacacaatatcatggttttaaagtgggcttggcccactttttttatggcaatttttcctcccaaaatagatgacaatggcaatgcaaccccaaactttggtgattaaaattggtacttctagcctagctcgtccggaaacgggtcagttggccctctccaccattgccgctttggtggaaactgtatgcaagttgatcggccaaggccatcgggtggtgctagtctcctctggagccataggagtaggttgtagtcgtttgggcctgacagaaaggccaaaaaaaatggccttaaagcaggcgatcgccgctgtgggccagggcagattaatgcggacctatgacgacctttttagtagcctccggcaacccattgcccaaattttgctcaccagacgggaattaattgagcgtaccgcctatgtcaacgcctacaacacgttccaagctctgtttgagttgggggtgattgccattgtcaatgaaaacgacacggtggcgatcgatgaactaaaatttggcgacaatgatactttgtcggctttagtggccagcttggtggaagcggattggctatttttgctcactgacgttgaccgcctttattccagtgacccccgtttagatcccgatgcctatcccattcccctagtcaaagccgcggaattagcccaattacaagtccgcaccgatagcactggttccgcctggggcaccggcggcatggccaccaaaatcaccgctgcccgcattgctacgggatcaggagtccgcaccgtcatcacccatggccaaaaacctgagcaaattttagccatcctccagggagctaatttgggcactcagtttgaagcccaaccccgctccgatatgctcgtaaacgttggatcgcctatggtttagtgcccaccgggaaaattttcattgacgctggggcggtccaggccctcaaagctaggggcaaatccctattggcgatcggggtggttgccctagaaggggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctccc

SEQ ID NO: 2 - DNA, 7381 bp, Artificial
  misc_difference (1)..(7381): Expression Vector
gtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgatgataagctgtcaaacatgagaattcttgaagacgaaagggcctcttgcgggatatcatccattccgacagcatcgccagtcactatggcgtgctgctagccgccacacaatatcatggttttaaagtgggcttggcccactttttttatggcaatttttcctcccaaaatagatgacaatggcaatgcaaccccaaactttggtgattaaaattggtacttctagcctagctcgtccggaaacgggtcagttggccctctccaccattgccgctttggtggaaactgtatgcaagttgatcggccaaggccatcgggtggtgctagtctcctctggagccataggagtaggttgtagtcgtttgggcctgacagaaaggccaaaaaaaatggccttaaagcaggcgatcgccgctgtgggccagggcagattaatgcggacctatgacgacctttttagtagcctccggcaacccattgcccaaattttgctcaccagacgggaattaattgagcgtaccgcctatgtcaacgcctacaacacgttccaagctctgtttgagttgggggtgattgccattgtcaatgaaaacgacacggtggcgatcgatgaactaaaatttggcgacaatgatactttgtcggctttagtggccagcttggtggaagcggattggctatttttgctcactgacgttgaccgcctttattccagtgacccccgtttagatcccgatgcctatcccattcccctagtcaaagccgcggaattagcccaattacaagtccgcaccgatagcactggttccgcctggggcaccggcggcatggccaccaaaatcaccgctgcccgcattgctacgggatcaggagtccgcaccgtcatcacccatggccaaaaacctgagcaaattttagccatcctccagggagctaatttgggcactcagtttgaagcccaaccccgctccgatatgctcgtaaacgttggatcgcctatggtttagtgcccaccgggaaaattttcattgacgctggggcggtccaggccctcaaagctaggggcaaatccctattggcgatcggggtggttgccctagaaggggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagagtttcaccgtcatcaccgaaacgcgcgagacgaaagggcctcgtgatacgctatttttataggttaatgtcatgataataatggttcttagacgtcaggtggcacttttcggggaaatgtgcgcggaacccgtatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggtggagggggccgtaggagtagcccgcaccattcaacgatcaaccgatggccgttatgtggcggtgtcagcccggggtaatttttattccacctgggcaccgggacaaacggaatggactccccataaccgcaattcttcccgacgcttacagaccatgggctatggcaaggacggtcaactatggctgttggcccgggggggacaactccagttcagcaccgatcccgatgcagaggaatggagcgatgtgattgctccccaggataaaggtagttggggtctgctcgatctgtctttccgtacccctgaagaagtatgggtagcgggggccagcggtaacctcttgatgagtcaagacggggggcaaacctgggccaaggacactggggtagaagatattccagccaatctttatcgggtggtgttcctcagtccggaaaaaggatttgtgttggggcaggatgggattttgctcaaatataaccccagcaccgaggtggcaatggttccctaggcggctcacaaaatagtagactagactctacttgctttgcatttgtcagtcaatgttgttttgaaaaattgaaggagaacacaaaatgtcagggactaccggcgagcgtccattttccgatattgtcaccagcattcgctactgggtgatccacagcatcaccatcccgatgttgtttattgctggttggttgtttgtcagcacgggcttagcctacgatgcttttggcactccccgccccgatgaatatttcacccagacccgtcaagagttgcccattctccaggaacgctacgacattaatcaggaaattcaagagtttaatcaataaaacatttaattgttcttttttagttggtaattaacaatggcaacccaaaatcctaatcaaccggttacttatcccatttttacggtgcgctggctggcggttcacaccctggcggtgccctctgtcttctttgtcggggcgatcgccgcgatgcaatttattcaacgctaggagtttttcatggacagaaattcaaacccaaaccgccaaccggtggaattgaaccgcacttctttatacctgggtctattgttggtggctgtgttggggattttgttctccagctatttctttaactaaacttttttaatacgcaatttaggaggcatggtatgttcgcagaaggcagaatccctttgtgggtggtgggtgtagtggccggtattggcgccattggtgttctagggttatttttctacggagcctatgctggtttaggttcttccatgtaatcgagggctagagtcgacatgctacacctccggataataaatatatataaacgtatatagatttcataaagtctaacacactagacttatttacttcgtaattaagtcgttaaaccgtgtgctctacgaccaaaactataaaacctttaagaactttctttttttacaagaaaaaagaaattagataaatctctcatatcttttattcaataatcgcatccgattgcagtataaatttaacgatcactcatcatgttcatatttatcagagctcgtgctataattatactaattttataaggaggaaaacatatgccaccatcatcatcaccatactagtactagagatctcgagaagatggactacaaggacgacgatgacaagcatgaggattacaaagacgatgacgataagcaaggtgactacaaagacgatgatgacaaatgagttaagggatgca

SEQ ID NO: 3 - DNA, 1817 bp, Artificial
  misc_difference (1)..(1817): Expression Vector
gtcgacctggtgagcaaaatttgggcaatgggttgccggaggctactaaaaaggtcgtcataggtccgcattaatctgccctggcccacagcggcgatcgcctgctttaaggccattttttttggcctttctgtcaggcccaaacgactacaacctactcctatggctccagaggagactagcaccacccgatggccttggccgatcaacttgcatacagtttccaccaaagcggcaatggtggagagggccaactgacccgtttccggacgagctaggctagaagtaccaattttaatcaccaaagtttggggttgcattgccattgtcatctattttgggaggaaaaattgccataaaaaaagtgggccaagcccactttaaaaccatgatattgtgtggcggctagcagcacgccatagtgactggcgatgctgtcggaatggatgatatcccgcaagaggccctttcgtcttcaagaattctcatgtttgacagcttatcatcgaattgcatctgaccgaattttacgtttccctgaataattctcatcaatcgtttcatcaattttatctttatactttatattttgtgcgttaatcaaatcataatttttatatgtttcctcatgatttatgtctttattattatagtttttattctctctttgattatgtctttgtatcccgtttgtattacttgatcctttaactctggcaaccctcaaaattgaatgagacatgctacacctccggataataaatatatataaacgtatatagatttcataaagtctaacacactagacttatttacttcgtaattaagtcgttaaaccgtgtgctctacgaccaaaactataaaacctttaagaactttctttttttacaagaaaaaagaaattagataaatctctcatatcttttattcaataatcgcatccgattgcagtataaatttaacgatcactcatcatgttcatatttatcagagctcgtgctataattatactaattttataaggaggaaaaaatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcaccaccaccaccaccactaagttaagggatgcataaactgcatcccttaacttgtttttgtcgac

SEQ ID NO: 4 - DNA, 1538 bp, Artificial
  misc_difference (1)..(1538): Expression Vector
gtcgacctggtgagcaaaatttgggcaatgggttgccggaggctactaaaaaggtcgtcataggtccgcattaatctgccctggcccacagcggcgatcgcctgctttaaggccattttttttggcctttctgtcaggcccaaacgactacaacctactcctatggctccagaggagactagcaccacccgatggccttggccgatcaacttgcatacagtttccaccaaagcggcaatggtggagagggccaactgacccgtttccggacgagctaggctagaagtaccaattttaatcaccaaagtttggggttgcattgccattgtcatctattttgggaggaaaaattgccataaaaaaagtgggccaagcccactttaaaaccatgatattgtgtggcggctagcagcacgccatagtgactggcgatgctgtcggaatggatgatatcccgcaagaggccctttcgtcttcaagaattctcatgtttgacagcttatcatcgaattgcatctgaccgaattttacgtttccctgaataattctcatcaatcgtttcatcaattttatctttatactttatattttgtgcgttaatcaaatcataatttttatatgtttcctcatgatttatgtctttattattatagtttttattctctctttgattatgtctttgtatcccgtttgtattacttgatcctttaactctggcaaccctcaaaattgaatgagacatgctacacctccggataataaatatatataaacgtatatagatttcataaagtctaacacactagacttatttacttcgtaattaagtcgttaaaccgtgtgctctacgaccaaaactataaaacctttaagaactttctttttttacaagaaaaaagaaattagataaatctctcatatcttttattcaataatcgcatccgattgcagtataaatttaacgatcactcatcatgttcatatttatcagagctcgtgctataattatactaattttataaggaggaaaaaatatgttagaccagcaaaccattaacatcatcaaagccactgttcctgtattgaaggagcatggcgttaccattaccacgactttttataaaaacttgtttgccaaacaccctgaagtacgtcctttgtttgatatgggtcgccaagaatctttggagcagcctaaggctttggcgatgacggtattggcggcagcgcaaaacattgaaaatttgccagctattttgcctgcggtcaaaaaaattgcagtcaaacattgtcaagcaggcgtggcagcagcgcattatccgattgtcggtcaagaattgttgggtgcgattaaagaagtattgggcgatgccgcaaccgatgacattttggacgcgtggggcaaggcttatggcgtgattgcagatgtgtttattcaagtggaagcagatttgtacgctcaagcggttgaacaccaccaccaccaccactaagttaagggatgcataaactgcatcccttaacttgtttttgtcgac

SEQ ID NO: 5 - DNA, 538 bp, Artificial
  misc_difference (1)..(538): Plasmid
gtcgacggcagtattttgttcctttggccaatggggcgatcggggaaaaatggcttgatctggcatttacgagaaaaatttttattttttaatgatttattttttcctattaaaatcttttttttacctttggaaaccaactgcaatctgagaaaccatcttgtttttttaaagaaatattattaatctgaaattcaagggaagttaatcaatgccaataattatctcgcattattaatccccctttatctatctggttgagttggatttagctgatagtttatcaccaaaataacaagcaaaatcaaatccaagcttaaacccaaaatcttacttcgtaattattccatatgcaccatcatcatcaccatactagtactagagatctcgagaagatggactacaaggacgacgatgacaagcatgaggattacaaagacgatgacgataagcaaggtgactacaaagacgatgatgacaaatgagttaagggatgcataaactgcatcccttaacttgtttttcgtgtgccgcatgc

SEQ ID NO: 6 - DNA, 7430 bp, Artificial
  misc_difference (1)..(7430): Plasmid
taagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgatgataagctgtcaaacatgagaattcttgaagacgaaagggcctcttgcgggatatcatccattccgacagcatcgccagtcactatggcgtgctgctagccgccacacaatatcatggttttaaagtgggcttggcccactttttttatggcaatttttcctcccaaaatagatgacaatggcaatgcaaccccaaactttggtgattaaaattggtacttctagcctagctcgtccggaaacgggtcagttggccctctccaccattgccgctttggtggaaactgtatgcaagttgatcggccaaggccatcgggtggtgctagtctcctctggagccataggagtaggttgtagtcgtttgggcctgacagaaaggccaaaaaaaatggccttaaagcaggcgatcgccgctgtgggccagggcagattaatgcggacctatgacgacctttttagtagcctccggcaacccattgcccaaattttgctcaccagacgggaattaattgagcgtaccgcctatgtcaacgcctacaacacgttccaagctctgtttgagttgggggtgattgccattgtcaatgaaaacgacacggtggcgatcgatgaactaaaatttggcgacaatgatactttgtcggctttagtggccagcttggtggaagcggattggctatttttgctcactgacgttgaccgcctttattccagtgacccccgtttagatcccgatgcctatcccattcccctagtcaaagccgcggaattagcccaattacaagtccgcaccgatagcactggttccgcctggggcaccggcggcatggccaccaaaatcaccgctgcccgcattgctacgggatcaggagtccgcaccgtcatcacccatggccaaaaacctgagcaaattttagccatcctccagggagctaatttgggcactcagtttgaagcccaaccccgctccgatatgctcgtaaacgttggatcgcctatggtttagtgcccaccgggaaaattttcattgacgctggggcggtccaggccctcaaagctaggggcaaatccctattggcgatcggggtggttgccctagaaggggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagagtttcaccgtcatcaccgaaacgcgcgagacgaaagggcctcgtgatacgctatttttataggttaatgtcatgataataatggttcttagacgtcaggtggcacttttcggggaaatgtgcgcggaacccgtatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggtggagggggccgtaggagtagcccgcaccattcaacgatcaaccgatggccgttatgtggcggtgtcagcccggggtaatttttattccacctgggcaccgggacaaacggaatggactccccataaccgcaattcttcccgacgcttacagaccatgggctatggcaaggacggtcaactatggctgttggcccgggggggacaactccagttcagcaccgatcccgatgcagaggaatggagcgatgtgattgctccccaggataaaggtagttggggtctgctcgatctgtctttccgtacccctgaagaagtatgggtagcgggggccagcggtaacctcttgatgagtcaagacggggggcaaacctgggccaaggacactggggtagaagatattccagccaatctttatcgggtggtgttcctcagtccggaaaaaggatttgtgttggggcaggatgggattttgctcaaatataaccccagcaccgaggtggcaatggttccctaggcggctcacaaaatagtagactagactctacttgctttgcatttgtcagtcaatgttgttttgaaaaattgaaggagaacacaaaatgtcagggactaccggcgagcgtccattttccgatattgtcaccagcattcgctactgggtgatccacagcatcaccatcccgatgttgtttattgctggttggttgtttgtcagcacgggcttagcctacgatgcttttggcactccccgccccgatgaatatttcacccagacccgtcaagagttgcccattctccaggaacgctacgacattaatcaggaaattcaagagtttaatcaataaaacatttaattgttcttttttagttggtaattaacaatggcaacccaaaatcctaatcaaccggttacttatcccatttttacggtgcgctggctggcggttcacaccctggcggtgccctctgtcttctttgtcggggcgatcgccgcgatgcaatttattcaacgctaggagtttttcatggacagaaattcaaacccaaaccgccaaccggtggaattgaaccgcacttctttatacctgggtctattgttggtggctgtgttggggattttgttctccagctatttctttaactaaacttttttaatacgcaatttaggaggcatggtatgttcgcagaaggcagaatccctttgtgggtggtgggtgtagtggccggtattggcgccattggtgttctagggttatttttctacggagcctatgctggtttaggttcttccatgtaatcgagggctagagtcgacggcagtattttgttcctttggccaatggggcgatcggggaaaaatggcttgatctggcatttacgagaaaaatttttattttttaatgatttattttttcctattaaaatcttttttttacctttggaaaccaactgcaatctgagaaaccatcttgtttttttaaagaaatattattaatctgaaattcaagggaagttaatcaatgccaataattatctcgcattattaatccccctttatctatctggttgagttggatttagctgatagtttatcaccaaaataacaagcaaaatcaaatccaagcttaaacccaaaatcttacttcgtaattattccatatgcaccatcatcatcaccatactagtactagagatctcgagaagatggactacaaggacgacgatgacaagcatgaggattacaaagacgatgacgataagcaaggtgactacaaagacgatgatgacaaatgagt

SEQ ID NO: 7 - DNA, 5676 bp, Artificial
  misc_difference (1)..(5676): Plasmid
ctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgttagaccaacaaaccgtagacaccagcaaagccactgttcctgtattgaaagagcatggcgtgaccattaccacgacgttttaccaaaatttgtttgccaaacatcctgaagtacgacctttgtttgacatgggtcgccaagcatctttggaacagcctaaggctttggcgatgacggttggggcggcggcacaaaacattgaaaatttacctgcaattttgcctgcagtacaaaaaattgccgtcaaacattgtcaagcaggcgtggcggcacgacattatccgattgtgggtcaagaattgttgggtgcgattaaagaattattgggtgatgcggcgaccgatgatattttggatgcgtggggcaaggcttatggcgtgattgccgatgtttttattcaagtggaagcggatttgtacgctcaagacgctgaa

SEQ ID NO: 8 - DNA, 377 bp, Artificial
  misc_difference (1)..(377): Synthesized DNA fragment
gtcgacggcagtattttgttcctttggccaatggggcgatcggggaaaaatggcttgatctggcatttacgagaaaaatttttattttttaatgatttattttttcctattaaaatcttttttttacctttggaaaccaactgcaatctgagaaaccatcttgtttttttaaagaaatattattaatctgaaattcaagggaagttaatcaatgccaataattatctcgcattattaatccccctttatctatctggttgagttggatttagctgatagtttatcaccaaaataacaagcaaaatcaaatccaagcttaaacccaaaatcttacttcgtaattattccatatgcaccatcatcatcaccatactagt

SEQ ID NO: 9 - DNA, 29 bp, Artificial
  misc_difference (1)..(29): forward primer
ggggggtcgactcgtaattaagtcgttaa

SEQ ID NO: 10 - DNA, 30 bp, Artificial
  misc_difference (1)..(30): reverse primer
gggggcatatgttttcctccttataaaatt

SEQ ID NO: 11 - DNA, 30 bp, Artificial
  misc_difference (1)..(30): forward primer
aacttgtttttcgtgtgcctattttttgtg

SEQ ID NO: 12 - DNA, 30 bp, Artificial
  misc_difference (1)..(30): reverse primer
ctacggagcctatgctggtttaggttcttc

SEQ ID NO: 13 - DNA, 31 bp, Artificial
  misc_difference (1)..(31): forward primer
aaaaagtcgacctggtgagcaaaatttgggc

SEQ ID NO: 14 - DNA, 42 bp, Artificial
  misc_difference (1)..(42): reverse primer
gctcctcgcccttgctcaccatattttttcctccttataaaa

SEQ ID NO: 15 - DNA, 72 bp, Artificial
  misc_difference (1)..(72): reverse primer
tttgatgatgttaatggtttgctggtctaacatattttttcctccttataaaattagtataattatagcacg

SEQ ID NO: 16 - DNA, 42 bp, Artificial
  misc_difference (1)..(42): forward primer
ttttataaggaggaaaaaatatggtgagcaagggcgaggagc

SEQ ID NO: 17 - DNA, 77 bp, Artificial
  misc_difference (1)..(77): reverse primer
aaaagtcgacaaaaacaagttaagggatgcagtttatgcatcccttaacttagtggtggtggtggtggtgcttgtac

SEQ ID NO: 18 - DNA, 53 bp, Artificial
  misc_difference (1)..(53): forward primer
ttttataaggaggaaaaaatatgttagaccagcaaaccattaacatcatcaaa

SEQ ID NO: 19 - DNA, 97 bp, Artificial
  misc_difference (1)..(97): reverse primer
aaaaagtcgacaaaaacaagttaagggatgcagtttatgcatcccttaacttagtggtggtggtggtggtgttcaaccgcttgagcgtacaaatctg

SEQ ID NO: 20 - DNA, 247 bp, Synechocystis sp. PCC 6803
atgtcagggactaccggcgagcgtccattttccgatattgtcaccagcattcgctactgggtgatccacagcatcaccatcccgatgttgtttattgctggttggttgtttgtcagcacgggcttagcctacgatgcttttggcactccccgccccgatgaatatttcacccagacccgtcaagagttgcccattctccaggaacgctacgacattaatcaggaaattcaagagtttaatcaataaa

SEQ ID NO: 21 - DNA, 135 bp, Synechocystis sp. PCC 6803
atggcaacccaaaatcctaatcaaccggttacttatcccatttttacggtgcgctggctggcggttcacaccctggcggtgccctctgtcttctttgtcggggcgatcgccgcgatgcaatttattcaacgctag

SEQ ID NO: 22 - DNA, 120 bp, Synechocystis sp. PCC 6803
atggacagaaattcaaacccaaaccgccaaccggtggaattgaaccgcacttctttatacctgggtctattgttggtggctgtgttggggattttgttctccagctatttctttaactaa

SEQ ID NO: 23 - DNA, 120 bp, Synechocystis sp. PCC 6803
atgttcgcagaaggcagaatccctttgtgggtggtgggtgtagtggccggtattggcgccattggtgttctagggttatttttctacggagcctatgctggtttaggttcttccatgtaa

SEQ ID NO: 24 - DNA, 850 bp, Bacillus subtilis
atgggcatttttagtatttttgtaatcagcacagttcattatcaaccaaacaaaaaataagtggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataa

SEQ ID NO: 25 - DNA, 84 bp, Artificial
  CDS (1)..(84): Synthesized DNA
gactacaaggacgacgatgacaagcatgaggattacaaagacgatgacgataagcaaggtgactacaaagacgatgatgacaaa

SEQ ID NO: 26 - protein, 28 aa, Artificial
DYKDDDDKHEDYKDDDDKQGDYKDDDDK

SEQ ID NO: 27 - DNA, 18 bp, Artificial
  CDS (1)..(18)
caccaccaccaccaccac

SEQ ID NO: 28 - protein, 6 aa, Artificial
HHHHHH

SEQ ID NO: 29 - DNA, 717 bp, Aequorea Victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 30 - DNA, 438 bp, Vitreoscilla spp.
atgttagaccaacaaaccgtagacaccagcaaagccactgttcctgtattgaaagagcatggcgtgaccattaccacgacgttttaccaaaatttgtttgccaaacatcctgaagtacgacctttgtttgacatgggtcgccaagcatctttggaacagcctaaggctttggcgatgacggttggggcggcggcacaaaacattgaaaatttacctgcaattttgcctgcagtacaaaaaattgccgtcaaacattgtcaagcaggcgtggcggcacgacattatccgattgtgggtcaagaattgttgggtgcgattaaagaattattgggtgatgcggcgaccgatgatattttggatgcgtggggcaaggcttatggcgtgattgccgatgtttttattcaagtggaagcggatttgtacgctcaagacgctgaa

SEQ ID NO: 31 - DNA, 978 bp, Zea mays
atgagtaaaagtgagcgtcgtgtttttctgctggattttgaaaagcccctctatgagctagaggaaaaaatcaatcaaatccgggaactagccgaggaaaaaaatgtggacgtgtccgaacagctcagtcaactagaaagtcgggctgaacagttgcgtcaggaaatttttagcaatctcaatccttcccagcggttacagttggcccgccatccccgccgtcctagtacgttggattatatccaagccattgcggatgattggtttgaaatgcacggcgatcgaggaggttatgacgatccggctttggtggggggagtggcccgcttggggactcgacctgtggtgattatgggccatcaaaagggaagggataccaaggataatgtggctcgcaattttggtatggcggcccccaatggttaccggaaggctctgcggttgatggaacacgctgatcgttttggtatgcctattattacttttattgatacccctggagcttgggccggcatcgatgcagaaaagttgggccaaggggaggcgatcgccgttaatttgcgggaaatgtttcggttagatgtccccattctctgcacagtgatcggggaggggggctccgggggtgcattgggtattggcgttggcgatcgggtattgatgctggaaaatgcggtgtacaccgtggctactccagaggcctgtgcggctattctttggaaggatgcgaaaaagtccgacaaagcggcgatcgccttgaaaattaccgccgatgatttagcgaagttacaaatcattgatgggattattcccgagccgaagggggccgcccacgccaatccattgggagccgctgctaagttgaaggaagctttattgttccatctcaataccttggctcaattaactccccaggaacgcaaacaattgcggtacgacaaattccggcatttgggtcaatttttagaaacggcggtg

SEQ ID NO: 32 - DNA, 462 bp, Zea mays
gtggctattaactttacggaactgcgggaattgttgggggtaatttcccaaagtaatatcacggagttcagtctaaaaagcggcgattttgaggtgtcagtccgtaaggatggtatggccggcggcatctccgttgtccctcaggcgatcgccccccagcctgccccggtggtttctgcctcagttccctcccccgaagttgcggccccatccccagcggatcaaaagtggacggcgatcgtttcccccatggtgggtactttttaccgagccccagccccggatgagcctccctttgtggaagtgggagatgcagtcagcaaaggccagggagtgtgcatcattgaagcaatgaagttaatgaatgaaattgaagcggaagtggcggggcaagtgatggagattgtggtggaaaatggggagccggtggaatacggtcaaaccctaatgtggattaaaccc

SEQ ID NO: 33 - DNA, 1344 bp, Zea mays
atgcaattcgccaaaattttaattgccaatcggggagagatcgccctacgcatcattcacagttgcgaagaattgggcattcccaccgtggcagtccactccaccatcgatcgccatgccctccacgtgcaactagccaacgagagtgtgtgcattgggccaccccccagcaataaaagctatctcaacattcccaatatcattgccgccgccctcacccgcaacgccactgccattcaccctggttacggatttttagcggaaaatgcccgctttgctgaaatttgtgccgaccatcaaattactttcatcggccccagccctgaagccatcaccgccatgggggataaatccaccgccaagaaaaccatgcaaaagtccggtgtgccctgtgtgcctggcagtccgggcttgattgagtcggaagcaacggccctaaaaattgcagcggaaattggttacccggtaattattaaagccacagctgggggcggcggtagggggatgcgcctcgtccaggaagaaaaggattttctcaaactgttccatgcggcccagggggaagcaggggcggcctttggcaatccaggggtttacctggaaaaattcattgaaaaaccccgccatattgagttccaaattttggccgatagccacggtaacgtagtgcacctgggggaaagggattgttccatccaacggcgacatcaaaagttactcgaagaagcccctagtccctttttgactccccacttgcggaaaaaaatgggggaggcggcggtgaaagcggccaaatccattaactacgtcggagccggcacagtggaatttttggtggatggtaacggtaatttctactttatggaaatgaacacccgcattcaggtggagcatccggttacagaaatgattaccggctatgacttgatttcggaacaaatccgcattgccatgggagaaaaactccgcttccgccagtccgacgtggaaattcgtggccatgccattgaatgccgcattaatgctgaagatcccaaacaaaattttcgcccccaccccggcaaaatcagtgcttatctcccccccggtggccccggagtccgcattgattcccacgtttacaccgactatgaaatccctccctactacgattccctgatcggcaaattaattgtttgggctggcgatcgcccttcggccatcaagagaatgcagagagccttgcgggaatgcgccatcactggagtacccaccacattggaatttcaccagcgcatactacaaaccccagcttttctggccggggatgtgtacaccaatttcatcgaagagcatttaacaccc

SEQ ID NO: 34 - DNA, 978 bp, Zea mays
atgtctctatttgattggtttgccaaccgtgaaaaagctgagcccccagtgctccagccccaatccccccaggaaagggaggtggccgatggtctctggactaagtgcccggcctgcggtgtgttgacctacaccaaggatctgcagggcaattggatggtgtgtgtcgaatgtgggcaccatctccgggtggacagtgacgagcggattcgccagttgattgacgccaagacctggcaacccatcaatgaacatttgcggcccaccgatcccctcaaatttaaagaccgcaaatcctataaagaccgcatccgcgatacccaaaaagcaacagatttgacggacgcagtgcaaactggccacggtcgccttgacggtctaccgatcgccttgggggtaatggatttccggtttatgggcggtagtatgggctccgtggtaggggaaaaactatgccgcctaattgagtacgccactttggaaagattgcccgtggtaattatttgtgcctccggtggagccagaatgcaggaaggcatgctgagcctaatgcaaatggccaaaatttctggagctttgcaaaaccatcgggagcaaaagttactttacatccctgtgctcacccatcccaccactggcggggtgactgccagctttgccatgttgggggatttgattttagcggaacccaaagccaccatcggttttgcggggcggcgagtgattgagcaaacattgcgggaaaagttacccgacgattttcaaacttccgaatatttactacaccacggttttgtcgatgccattgtgccccgtccccaactgaaaaggactttggctcagttgattagcctacatcaacccttttatcccattttgccccccctcaatgctgactccaatcaggtgaacccagagttagtgctcagccatacggccctggcggtggataatcaaatttccgtcaatcaagatggt

SEQ ID NO: 35 - DNA, 675 bp, Synechocystis sp. PCC 6803
gtggattccgagattaatcatcgtggtggtttgagtgctccccgcccaagggaaacgtcacttaatttagctctctaccggggcttgaaatggggggtggtgcggccactgctccatggattgttccaggcccaggtatatggtcaggaattggtgccaacccgggggccggccttggtggtgagcaaccatgccagttattttgaccccccatttttgtcctgtgccatggcccggccggtggcctttatggccaaggaagagttatttaatgtgcccctgctgggtccagccattcgcctctatggggcctatccagtcaaacggggcagtggcgatcggggagcattgcgggccgccttgacggcgctgggggatggttggttagtgggggtctttctggagggaaccagaacaaaggatggccgcattcaccagccaaaattgggggctgccatgattgcagctaaagcccaagtgcccattattcccgtcagcctagggggagtagagcaaatttttcagcccggttccccctggccccatcctgtgcctttaactattcgcattggtaaggcgatcgcccctccagtaaagaataggaaacccgaattggaagcggttactaaagcttgccaagcccaaattcacgagatgctggatttaggcagggat

SEQ ID NO: 36 - DNA, 801 bp, Synechocystis sp. PCC 6803
atgggattatttgaccgtttaggccgcgtcgtccgggctaacctcaacgacctcgtcagtaaagctgaagatccagaaaaagttctggaacaggccgtgatcgatatgcaggaagacctggtgcaactccgccaggccgttgcccgcacgatcgccgaagaaaaacgaacagagcaacgtcttaaccaagacacccaggaagccaagaaatgggaagaccgggcaaaattagccctcaccaatggggaagaaaatttggctagggaagccctggcccgcaaaaaaagtctgacagatacggcggcggcgtaccaaacccagctagcccaacagaggaccatgtcggagaatctccgtcgcaacctcgcggctctagaagcaaaaatttccgaagctaaaaccaagaaaaatatgttgcaggccagggccaaagcggccaaggctaatgctgaactgcagcaaaccctcgggggcttaggtactagcagtgctactagtgcttttgaacggatggagaacaaggtactggatatggaagccacttcccaagcggcaggggagttagccggctttggcatcgagaaccagtttgcccagttggaagccagcagtggggtagaagacgagttggccgccctgaaagcttccatggccggtggagcattaccgggaacctctgccgctacgccccaactcgaagcggctcctgttgattcctcagtaccagctaataatgccagtcaagatgatgcggtgattgaccaggaattggacgacctacgtcgtcggttaaataatctg

SEQ ID NO: 37 - DNA, 1005 bp, Arabidopsis thaliana
atggagagtttcccgatcatcaatctcgagaagcttaatggagaagagagagcaatcactatggagaagatcaaagacgcttgtgaaaactggggcttctttgagtgtgtgaaccatgggatttcactcgagcttttggacaaagtggagaagatgaccaaggaacattacaagaagtgcatggaagagagattcaaggaatcgattaagaacagaggtcttgactctcttcgctctgaagtcaacgacgttgactgggaatccactttctacctcaagcaccttcccgtctctaatatctccgatgtccctgatctcgacgacgattacagaacgttaatgaaagacttcgccggaaagatagagaagttgtcggaggagctactggatctgctgtgcgagaatctcggtttagagaagggttatttaaaaaaggtgttttacgggtcgaaaagaccgacttttggaaccaaagtcagcaattatccaccttgtcctaatccggacctagtcaagggtctccgagcccacaccgacgccggcggcatcatcctcctcttccaagacgacaaagtcagtggacttcagcttcttaaagacggcgagtgggtcgatgttcctccggttaagcattcaatcgtcgttaatctcggcgatcaacttgaggtgataaccaatgggaagtacaagagtgtggaacatagagtgctatctcagacagacggagaaggaagaatgtcgatcgcatcattctataatccgggaagcgactctgttatttttccggcgccggagctgatcggaaaagaagcagagaaggagaagaaagagaactatccgagatttgtgtttgaagattacatgaaactctactctgctgtcaagtttcaggccaaggaaccaaggtttgaagccatgaaagctatggagacaactgtggccaacaatgttggaccattggccactgcgcttgcggccgcactcgagcaccaccaccaccaccac

SEQ ID NO: 38 - DNA, 1521 bp, Arabidopsis thaliana
atggtggcttttgcaacagagaagaagcaagatctgaatctattgtctaaaatcgcctccggtgacggtcacggcgagaattcctcttatttcgatggttggaaagcttatgaagaaaacccatttcacccaattgatagacctgacggagttattcaaatgggtctcgctgaaaatcagctttgtggagatttgatgcgtaaatgggttttaaaacatccagaagcttcgatttgtacatcagaaggtgtgaatcaattcagtgacattgccatttttcaagattatcatggcttgcctgaattcagacaagctgtagcgaaatttatggagaagactagaaataacaaagttaagtttgatcctgaccggattgttatgagcggcggcgcaaccggagcacacgagacggttgctttctgtttagctaatcccggcgatggtttcttagttccaaccccttattatccagggtttgatagagatttgagatggagaaccggagtgaatcttgtaccggttacttgtcatagctctaatgggttcaagattacggtggaagccttggaagctgcttacgaaaacgcgagaaaatcgaatattccggttaagggtttacttgtaaccaatccttcaaacccgcttggtacgacgttagaccgggaatgtttgaagtctcttgttaacttcactaatgacaaagggattcatcttattgctgatgagatttatgctgctactacttttggtcaatccgagttcataagtgttgcggaagtaatcgaggagatcgaagattgtaaccgcgatttgatacatattgtgtatagtctatctaaagatatgggtctgcctggtttaagagttggtatagtatactcttacaatgacagggtggttcagatcgcaaggaaaatgtcgagtttcggtcttgtttcgtcacaaacgcagcatttgatcgctaaaatgttatccgatgaagagtttgtagacgagtttatccgcgagagcaaattgcggttagctgcaaggcacgctgagataaccaccggtttagatggtttagggattggttggttaaaggccaaagccggtttgttcttgtggatggatttaagaaatcttttgaagacagcaacgtttgattcggaaaccgaactatggcgtgtgattgttcaccaagtgaagctcaacgtgtctccaggcggttcgttccattgccatgaaccgggatggtttagagtatgttttgcgaatatggaccataagacgatggagacagctctagagaggattagagtgttcactagccaacttgaggaggagactaaaccgatggctgcaacaactatgatggctaaaaagaagaagaagtgttggcagagtaacctcaggttaagctttagtgacacgaggcggttcgatgatggcttcttctcgcctcattcgcctgtgccgccttctccgctagtccgtgcacagactcttgcggccgcactcgagcaccaccaccaccaccac

SEQ ID NO: 39 - DNA, 1470 bp, Aspergillus terreus
atgaccaaacaatctgcggacagcaacgcaaagtcaggagttacgtccgaaatatgtcattgggcatccaacctggccactgacgacatcccttcggacgtattagaaagagcaaaataccttattctcgacggtattgcatgtgcctgggttggtgcaagagtgccttggtcagagaagtatgttcaggcaacgatgagctttgagccgccgggggcctgcagggtgattggatatggacagaaactggggcctgttgcagcagccatgaccaattccgctttcatacaggctacggagcttgacgactaccacagcgaagcccccctacactctgcaagcattgtccttcctgcggtctttgcagcaagtgaggtcttagccgagcagggcaaaacaatttccggtatagatgttattctagccgccattgtggggtttgaatctggcccacggatcggcaaagcaatctacggatcggacctcttgaacaacggctggcattgtggagctgtgtatggcgctccagccggtgcgctggccacaggaaagctcctcggtctaactccagactccatggaagatgctctcggaattgcgtgcacgcaagcctgtggtttaatgtcggcgcaatacggaggcatggtaaagcgtgtgcaacacggattcgcagcgcgtaatggtcttcttgggggactgttggcccatggtgggtacgaggcaatgaaaggtgtcctggagagatcttacggcggtttcctcaagatgttcaccaagggcaacggcagagagcctccctacaaagaggaggaagtggtggctggtctcggttcattctggcatacctttactattcgcatcaagctctatgcctgctgcggacttgtccatggtccagtcgaggctatcgaaaaccttcaggggagataccccgagctcttgaatagagccaacctcagcaacattcgccatgttcatgtacagctttcaacggcctcgaacagtcactgtggatggataccagaggagagacccatcagttcaatcgcagggcagatgagtgtcgcatacattctcgccgtccagctggtcgaccagcaatgtcttttgtcccagttttctgagtttgatgacaacctggagaggccagaagtttgggatctggccaggaaggttacttcatctcaaagcgaagagtttgatcaagacggcaactgtctcagtgcgggtcgcgtgaggattgagttcaacgatggttcttctattacggaaagtgtcgagaagcctcttggtgtcaaagagcccatgccaaacgaacggattctccacaaataccgaacccttgctggtagcgtgacggacgaatcccgggtgaaagagattgaggatcttgtcctcggcctggacaggctcaccgacattagcccattgctggagctgctgaattgccccgtgaaatcgccactggta

SEQ ID NO: 40 - DNA, 1167 bp, Zymomonas mobilis
atggcttcttcaactttttatattcctttcgtcaacgaaatgggcgaaggttcgcttgaaaaagcaatcaaggatcttaacggcagcggctttaaaaatgcgctgatcgtttctgatgctttcatgaacaaatccggtgttgtgaagcaggttgctgacctgttgaaagcacagggtattaattctgctgtttatgatggcgttatgccgaacccgactgttaccgcagttctggaaggccttaagatcctgaaggataacaattcagacttcgtcatctccctcggtggtggttctccccatgactgcgccaaagccatcgctctggtcgcaaccaatggtggtgaagtcaaagactacgaaggtatcgacaaatctaagaaacctgccctgcctttgatgtcaatcaacacgacggctggtacggcttctgaaatgacgcgtttctgcatcatcactgatgaagtccgtcacgttaagatggccattgttgaccgtcacgttaccccgatggtttccgtcaacgatcctctgttgatggttggtatgccaaaaggcctgaccgccgccaccggtatggatgctctgacccacgcatttgaagcttattcttcaacggcagctactccgatcaccgatgcttgcgccttgaaggctgcgtccatgatcgctaagaatctgaagaccgcttgcgacaacggtaaggatatgccagctcgtgaagctatggcttatgcccaattcctcgctggtatggccttcaacaacgcttcgcttggttatgtccatgctatggctcaccagttgggcggctactacaacctgccgcatggtgtctgcaacgctgttctgcttccgcatgttctggcttataacgcctctgtcgttgctggtcgtctgaaagacgttggtgttgctatgggtctcgatatcgccaatctcggtgataaagaaggcgcagaagccaccattcaggctgttcgcgatctggctgcttccattggtattccagcaaatctgaccgagctgggtgctaagaaagaagatgtgccgcttcttgctgaccacgctctgaaagatgcttgtgctctgaccaacccgcgtcagggtgatcagaaagaagttgaagaactcttcctgagcgctttccatcatcatcatcatcat

SEQ ID NO: 41 - DNA, 1722 bp, Zymomonas mobilis
  CDS (1)..(1722)
  misc_feature (622)..(622): n is a, c, g, or t
atgagttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgaccacgctgctggtcacgtgttgcatcacgctcttggcaaaaccgactatcactatcagttggaaatggccaagaacatcacggccgccgctgaagcgatttataccccggaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccnaccgcgacaaagttgccgtcctcgtcggcagcaagctgcgcgcagctggtgctgaagaagctgctgtcaaatttgctgatgctcttggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtctttaacgactactccaccactggttggacggatattcctgatcctaagaaactggttctcgctgaaccgcgttctgtcgtcgttaacggcattcgcttccccagcgtccatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacattggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctccaccaccaccaccaccac

SEQ ID NO: 42 - protein, 574 aa, Zymomonas mobilis
  misc_feature (208)..(208): The ′Xaa′ at location 208 stands for Asn, Asp, His, or Tyr.
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIAXRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLLHHHHHH

SEQ ID NO: 43 - DNA, 214 bp, Bacillus subtilis
tcgtaattaagtcgttaaaccgtgtgctctacgaccaaaactataaaacctttaagaactttctttttttacaagaaaaaagaaattagataaatctctcatatcttttattcaataatcgcatccgattgcagtataaatttaacgatcactcatcatgttcatatttatcagagctcgtgctataattatactaattttataaggaggaaaa

SEQ ID NO: 44 - DNA, 46 bp, Bacillus subtilis
agttaagggatgcataaactgcatcccttaacttgtttttcgtgtg

(19) United States
(12) Patent Application Publication
(10) Pub. No.: US 2011/0124088 A1
(43) Pub. Date: May 26, 2011

(54) Expression vector for expressing recombinant protein in Cyanobacterium

(76) Inventors: Pei-Fen Yang, Hsinchu (TW); Hui-Pin Chen, Hsinchu (TW); Hsiao-Ming Wan, Hsinchu (TW)

(21) Appl. No.: 12/655,599

(22) Filed: Dec. 31, 2009

(30) Foreign Application Priority Data
Nov. 20, 2009 (TW) 98139450

(51) Int. Cl. C12N 1/21; C12N 15/63

(52) U.S. Cl. 435/252.3; 435/320.1; 435/252.33

(57) ABSTRACT

The present invention provides a universal vector for expressing a protein in a Cyanobacterium that includes an erythromycin promoter and a homologous recombination DNA fragment, and further provides a transformed E. coli and a genetically modified Cyanobacterium. In addition, the present invention also provides a method for expressing a protein in a Cyanobacterium that includes steps of inserting a gene of a specific protein into the universal vector and transforming the vector into a Cyanobacterium, so as to express the specific protein.

### BACKGROUND OF THE INVENTION

1. Field of the Invention

The present invention relates to a vector for expressing a recombinant protein in a Cyanobacterium, more particularly to a vector for expressing a recombinant protein in Synechocystis sp. PCC 6803.

2. Description of Related Art

Cyanobacteria and bacteria all belong to prokaryotes. However, Cyanobacteria that perform photosynthesis to produce oxygen as higher plants are an autotrophic organism. Cyanobacteria can be found in the ocean or the fresh water due to the widespread habitat. In order to adapt to different environments, Cyanobacteria develop various mechanisms and structures. Since the differentiation of the prokaryotes is easier than that of the higher plants, the results obtained from analyses of the mechanisms, such as photosynthesis, nitrogen fixation, the formation of heterocysts and the cycle time, of the Cyanobacteria can be directly employed in plants.

Synechocystis sp. PCC 6803 was first purified in 1968 and became a main strain in researching Cyanobacteria for scientists because its growth conditions is broad and it is able to perform photosynthesis and heterotrophically grow with organic carbons (JOHN G. K. Williams, METHODS IN ENZYMOLOGY, VOL. 167, p. 766-778, 1988; Abhay K. Singh and Louis A. Sherman, JOURNAL OF BACTERIOLOGY, Vol. 187, No. 7, p. 2368-2376, 2005). In 1980, it was found that Synechocystis sp. PCC 6803 is able to perform natural transformation and is able to perform homologous recombination with a foreign DNA at its chromosome. Kufryk et al. demonstrates that high transformation efficiency in Synechocystis sp. PCC 6803 can be simply achieved by natural transformation (Galyna I. Kufryk, Monika Sachet, Georg Schmetterer, Wim F. J. Vermaas, FEMS Microbiology Letters, 206 (2002) 215-219). In 1996, 3,168 genes of Synechocystis sp. PCC 6803 was successfully sequenced (Masahiko Ikeuchi & Satoshi Tabata, Photosynthesis Research, 70: 73-83, 2001). Therefore, some specific sites, such as the core gene psbA for photosystem II, at the chromosome of Synechocystis sp. PCC 6803 are then used in protein expressions. Since Cyanobacteria have multiple copy numbers of psbA genes, the situation that one copy number of psbA gene is replaced with a foreign gene does not affect the growth of Cyanobacteria. The psbA promoter is often used to drive other gene expressions in Synechocystis sp. PCC 6803.

Studies of Cyanobacteria and related plasmid systems have been disclosed in prior arts. For example, Szalay et al. (U.S. Pat. No. 4,778,759 and WO 8400381) disclose a plasmid system having a foreign DNA stably inserted into the chromosome. However, Szalay et al. only provide genetic engineering in the Cyanobacteria and do not develop this system to be employed in the protein expression.

Further, Woods et al. (US 20020042111) disclose a plasmid system for producing ethanol in Cyanobacteria, in which the plasmid is a shuttle vector having gene fragments encoding pyruvate decarboxylase and alcohol dehydrogenase and a rbcLS promoter of the Cyanobacteria.

In addition, Vaeck et al., (U.S. Pat. No. 5,516,693 and U.S. Pat. No. 6,335,008) disclose a plasmid system for expressing a recombinant protein, Bacillus thruingiensis endotoxin (abbreviated as Bt8 toxin), in the Cyanobacteria, in which the promoter in this plasmid system is mainly from rbcL promoter and psbA promoter of the Cyanobacteria and the recombinant gene is introduced into the Cyanobacteria by homologous recombination to express Bt8 toxin.

However, the genes encoding the recombinant proteins in the above plasmids cannot be optionally changed for the desired recombinant proteins, and when genes encoding other proteins are inserted into the above plasmids, the genes cannot be effectively driven. Thus, when specific proteins are required to be expressed in Cyanobacteria for researches, the plasmids have to be re-designed. It is very inconvenient in research and use. As a result, there is an urgent demand to provide a universal vector in a Cyanobacterium, in which the gene encoding a protein can be optionally changed and various gene expressions can be effectively driven.

### SUMMARY OF THE INVENTION

The Applicant surprisingly found that an erythromycin promoter has excellent effect on driving gene expressions in Cyanobacteria, and further completes the present invention.

The present invention provides a novel expression vector system in a Cyanobacterium. This expression vector can be used to produce the desired protein in the Cyanobacterium, and the desired protein can be further purified.

In one aspect, the present invention provides an expression vector for expressing a protein in a Cyanobacterium that includes a protein expression cassette and a homologous recombination DNA fragment flanking the protein expression cassette, wherein the protein expression cassette includes an erythromycin promoter. In one embodiment of the present invention, examples of the homologous recombination DNA fragment include a psbE gene (SEQ ID NO. 20), a psbF gene (SEQ ID NO. 21), a psbL gene (SEQ ID NO. 22), a psbJ gene (SEQ ID NO. 23), DNA fragments thereof and a combination thereof. The expression vector of the present invention with the homologous recombination DNA fragment can be inserted into the chromosome of the Cyanobacterium by homologous recombination and expresses the protein.

According to the present invention, the protein expression cassette in the expression vector can further include at least one restriction site, such as AatI, ApaI, AscI, AspI, AvaI, BamHI, BglII, EagI, EcoNI, KpnI, MfeI, NspV, PacI, MunI, NspI, PmeI, PmlI, SfuI, SfiI, StuI, SwaI, NdeI, SalI, SpeI, XboI, XhoI, XmaII or a combination thereof.

According to the present invention, the protein expression cassette in the expression vector includes a terminator. Examples of the terminator include an erythromycin terminator (SEQ ID. NO. 44) and stop codons. Moreover, the protein expression cassette in the expression vector of the present invention further includes a selection marker gene, such as an erythromycin-resistance gene (SEQ ID NO. 24).

In one embodiment of the present invention, the protein expression cassette in the expression vector includes a gene encoding a protein, wherein the gene encoding the protein is inserted into the restriction site, and the encoded protein can be an endogenous protein or a heterologous protein. Examples of the gene encoding the protein include a gene encoding enhanced green fluorescent protein (EGFP), a gene encoding Vitreoscilla hemoglobin (VHb), genes encoding subunits of acetyl-CoA carboxylase (accA, accB, accC and accD), a gene encoding lysophosphatidic acid acyltransferase (LPAAT), a gene encoding chloroplast membrane-associated protein (VIPP1), a gene encoding 1-aminocyclopropane-1-carboxylic acid oxidase (ACO), a gene encoding 1-aminocyclopropane-1-carboxylic acid synthase (ACS), a gene encoding cis-aconitate decarboxylase (CAD), a gene encoding alcohol dehydrogenase (ADH) and a gene encoding pyruvate decarboxylase (PDC).

The expression vector of the present invention can further include a gene encoding a tag. Examples of the tag include a series of six histidine residues (also refer to 6× histidine tag herein) (SEQ ID NO. 28) and flag 3 tag (SEQ ID NO. 26). The tag can be designed at the C-terminus and/or the N-terminus of the protein to be expressed if need be. That is, the gene encoding the tag can be located at 3′ end and/or 5′ end of the gene encoding the protein in favor of analysis and/or purification of the recombinant protein expressed in the Cyanobacterium.

According to the present invention, examples of the Cyanobacterium include transformable strains of the following strains: Synechocystis sp., Synechococcus spp., Microcystis aeruginosa, Prochlorococcus marinus and Nostoc punctiforme.

In another aspect, the present invention provides an expression vector for expressing a protein that has been deposited at Food Industry Research and Development Institute (331 Shih-Pin Road, Hsinchu, 300 Taiwan, R. O. C.) on Sep. 28, 2009 and has been given the BCRC Accession No. BCRC 940573. The expression vector has also been deposited under Budapest Treaty at DSMZ-DEUTSCHE SAMMLUNG VON MIKROORGANISMEN UND ZELLKULTUREN GmbH (Inhoffenstr. 7 B, D-38124 Braunschweig, Germany) on Oct. 1, 2009 and has been given the DSMZ Accession No. DSM 22996 by the International Depositary Authority. Both biological depositary materials were subjected to the viability test and both were passed.

In still another aspect, the present invention provides uses of the above expression vectors in expressing proteins.

In yet another aspect, the present invention provides a transformed Escherichia coli (E. coli) having an expression vector, which includes a protein expression cassette and a homologous recombination DNA fragment. In another aspect of the present invention, when an expression vector of the present invention having a protein expression cassette and a homologous recombination DNA fragment is transformed into a Cyanobacterium, the homologous recombination DNA fragment allows the expression vector to be inserted into the chromosome of the Cyanobacterium by homologous recombination, so as to further provide a genetically modified Cyanobacterium, wherein the genetically modified Cyanobacterium can express the desired protein.

In the embodiment of the transformed Escherichia coli or the genetically modified Cyanobacterium, the protein expression cassette includes an erythromycin promoter, and examples of the homologous recombination DNA fragment include a psbE gene (SEQ ID NO. 20), a psbF gene (SEQ ID NO. 21), a psbL gene (SEQ ID NO. 22), a psbJ gene (SEQ ID NO. 23), DNA fragments thereof and a combination thereof.

In another embodiment, the protein expression cassette of the expression vector in the transformed Escherichia coli or the genetically modified Cyanobacterium of the present invention includes at least one restriction site, such as AatI, ApaI, AscI, AspI, AvaI, BamHI, BglII, EagI, EcoNI, KpnI, MfeI, NspV, PacI, MunI, NspI, PmeI, PmlI, SfuI, SfiI, StuI, SwaI, NdeI, SalI, SpeI, XboI, XhoI, XmaII or a combination thereof.

In the present invention, the protein expression cassette of the expression vector in the transformed Escherichia coli or the genetically modified Cyanobacterium includes a terminator. Examples of the terminator include an erythromycin terminator (SEQ ID. NO. 44) and stop codons. Moreover, the protein expression cassette further includes a selection marker gene, such as an erythromycin-resistance gene (SEQ ID NO. 24).

In one embodiment of the present invention, the protein expression cassette in the transformed Escherichia coli or the genetically modified Cyanobacterium further includes a gene encoding a protein, wherein the gene encoding the protein is inserted into the restriction site, and the encoded protein can be an endogenous protein or a heterologous protein. Examples of the gene encoding the protein include a gene encoding EGFP, a gene encoding VHb, genes encoding subunits of acetyl-CoA carboxylase (accA, accB, accC and accD), a gene encoding lysophosphatidic acid acyltransferase, a gene encoding chloroplast membrane-associated protein, a gene encoding 1-aminocyclopropane-1-carboxylic acid oxidase, a gene encoding 1-aminocyclopropane-1-carboxylic acid synthase, a gene encoding cis-aconitate decarboxylase, a gene encoding alcohol dehydrogenase and a gene encoding pyruvate decarboxylase.

In one embodiment of the transformed Escherichia coli or the genetically modified Cyanobacterium of the present invention, the protein expression cassette can further include a gene encoding a tag. Examples of the tag include 6× histidine tag (SEQ ID NO. 28) and flag 3 tag (SEQ ID NO. 26). The tag can be designed at the C-terminus and/or the N-terminus of the protein to be expressed if need be. That is, the gene encoding the tag can be located at 3′ end and/or 5′ end of the gene encoding the protein in favor of analysis and/or purification of the recombinant protein expressed in the Cyanobacterium.

According to the present invention, examples of the genetically modified Cyanobacterium include transformable strains of the following strains: Synechocystis sp., Synechococcus spp., Microcystis aeruginosa, Prochlorococcus marinus and Nostoc punctiforme.

In another aspect, the present invention provides an Escherichia coli or a Cyanobacterium having an expression vector for expressing a protein, wherein the expression vector has been deposited at Food Industry Research and Development Institute (331 Shih-Pin Road, Hsinchu, 300 Taiwan, R. O. C.) on Sep. 28, 2009 and has been given the BCRC Accession No. BCRC 940573. The expression vector has also been deposited under Budapest Treaty at DSMZ-DEUTSCHE SAMMLUNG VON MIKROORGANISMEN UND ZELLKULTUREN GmbH (Inhoffenstr. 7 B, D-38124 Braunschweig, Germany) on Oct. 1, 2009 and has been given the DSMZ Accession No. DSM 22996 by the International Depositary Authority. Both vectors were subjected to the viability test and were passed.

In another aspect, the present invention provides uses of the above genetically modified Cyanobacteria in expressing proteins.

In still one aspect, the present invention provides a method for expressing a protein in a Cyanobacterium that includes transforming one of the above expression vectors into a Cyanobacterium to express the protein. In this method, examples of the Cyanobacterium include transformable strains of the following strains: Synechocystis sp., Synechococcus spp., Microcystis aeruginosa, Prochlorococcus marinus and Nostoc punctiforme.

The gene sequence of the erythromycin promoter used in the present invention is SEQ ID. NO. 43 (T. J. Gryczan, G. Grandi, J. Hahn, R. Grandi and D. Dubnau, Nucleic acids research, Vol. 8, 6081-6097, 1980).

In addition, the nucleic acid sequences encoding flag 3 tag, 6× histidine tag, EGFP, VHb, accA, accB, accC, accD, lysophosphatidic acid acyltransferase, chloroplast membrane-associated protein, 1-aminocyclopropane-1-carboxylic acid oxidase, 1-aminocyclopropane-1-carboxylic acid synthase, cis-aconitate decarboxylase, alcohol dehydrogenase and pyruvate decarboxylase are SEQ ID. NO. 25, 27 and 29 to 41, respectively.

### BRIEF DESCRIPTION OF THE DRAWINGS

FIG. 1 shows the construction of pAC559em;

FIG. 2 shows the vector map of pAC-em;

FIG. 3 shows a flow chart for the construction of pAC-em-EGFP;

FIG. 4 shows a flow chart for the construction of pAC-em-VHb;

FIG. 5 shows results obtained from DNA electrophoresis for E. coli with the constructed expression vectors after PCR, wherein [M] represents DNA marker (1 Kb Ladder DNA marker; manufactured by Yestern Biotech Co., Ltd.); [1] represents pAC-em-EGFP; and [2] represents pAC-em-VHb;

FIG. 6A shows results obtained from colony PCR for Synechocystis sp. PCC 6803 containing pAC-em-EGFP, wherein [M] represents DNA marker (1 Kb Ladder DNA marker); [1] represents pAC-em-EGFP vector as a positive control; and [2]-[10] represent single colonies and the DNA fragment is 1954 bp;

FIG. 6B shows results obtained from colony PCR for Synechocystis sp. PCC 6803 containing pAC-em-VHb, wherein [M] represents DNA marker (1 Kb Ladder DNA marker); [1] represents pAC-em-VHb vector as a positive control; and [2]-[11] represent single colonies and the DNA fragment is 1675 bp;

FIG. 7A shows the western blot analysis for EGFP expressed in Synechocystis sp. PCC 6803 with the expression vector, pAC-em-EGFP;

FIG. 7B shows the western blot analysis for VHb expressed in Synechocystis sp. PCC 6803 with the expression vector, pAC-em-VHb;

FIG. 8A shows the western blot analysis for EGFP expressed in Synechocystis sp. PCC 6803/pAC-em-EGFP induced by erythromycin, wherein [M] represents protein marker (Prestained Protein Ladder, manufactured by MBI Fermentas); [1] represents Synechocystis sp. PCC 6803; [2] represents Synechocystis sp. PCC 6803/pAC559em; [3] represents Synechocystis sp. PCC6803/pAC-em-EGFP that was not induced by erythromycin; [4] represents Synechocystis sp. PCC 6803/pAC-em-EGFP induced by 0.1 μg/mL erythromycin; [5] represents Synechocystis sp. PCC 6803/pAC-em-EGFP induced by 0.2 μg/mL erythromycin; [6] represents Synechocystis sp. PCC 6803/pAC-em-EGFP induced by 0.5 μg/mL erythromycin; [7] represents BL21; and [8] represents BL21/pEGFP; and wherein [1]-[8] represent total proteins; and

FIG. 8B shows the western blot analysis for VHb expressed in Synechocystis sp. PCC 6803/pAC-em-VHb induced by erythromycin, wherein [M] represents protein marker (Prestained Protein Ladder); [1] represents Synechocystis sp. PCC 6803; [2] represents Synechocystis sp. PCC 6803/pAC559em; [3] represents Synechocystis sp. PCC 6803/pAC-em-VHb that was not induced by erythromycin; [4] represents Synechocystis sp. PCC 6803/pAC-em-VHb induced by 0.1 μg/mL erythromycin; [5] represents Synechocystis sp. PCC 6803/pAC-em-VHb induced by 0.2 μg/mL erythromycin; [6] represents Synechocystis sp. PCC 6803/pAC-em-VHb induced by 0.5 μg/mL erythromycin; and [7] represents DH5α/pET14b-VHb; and wherein [1]-[7] represent total proteins.

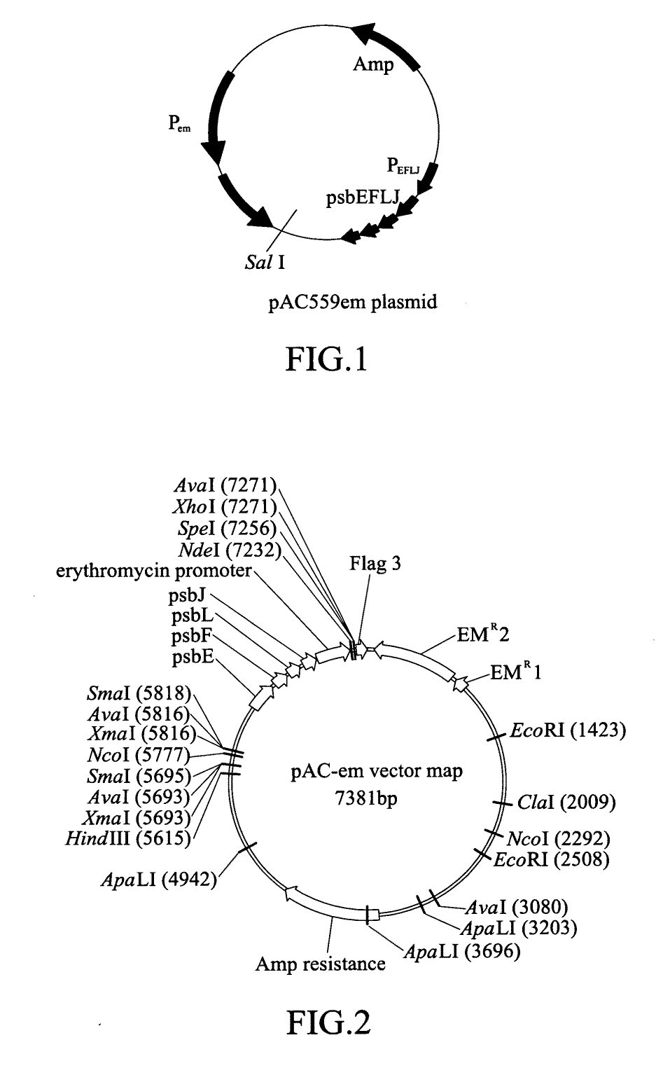

### DETAILED DESCRIPTION OF THE PREFERRED EMBODIMENTS

The following illustrative embodiments are provided to illustrate the disclosure of the present invention. These and other advantages and effects can be apparently understood by those in the art after reading the disclosure of this specification.

Herein, “em” in the names of the expression vectors represents the erythromycin promoter (SEQ ID. NO. 43).

TABLE 1
List of Expression Vectors

| Name of Expression Vector | SEQ ID NO. | Expressed Protein |
|---|---|---|
| pAC-em | 2 | — |
| pAC-em-EGFP | 3 | EGFP |
| pAC-em-VHb | 4 | VHb |

### Example 1

### Construction of pAC-em

The DNA fragment, SalI-psb promoter-multiple cloning site-erythromycin terminator-SphI (abbreviated as psb-Tem, SEQ ID NO. 8), was firstly designed, wherein the multiple cloning site included restriction sites, NdeI, SpeI, XhoI, AvaI and so on. The psb-Tem was synthesized by Invitrogen™ and was constructed into a plasmid pUC57 (manufactured by GeneDireX, Inc.). The thus-obtained plasmid was named pUC57-psb (SEQ ID NO. 5). The restriction enzyme reaction was performed on a plasmid pAC559em (SEQ ID NO. 1) and pUC57-psb with the restriction enzymes, SalI and SphI (manufactured by New England Biolabs, USA). After the restriction enzyme reaction, the psb-Tem and pAC559em were purified by Gel Extraction kit (Gel-M™ Gel Extraction System, manufactured by VIOGENE Co., Taiwan), and ligation was performed on the psb-Tem and pAC559em with a ratio of 3:1. The thus-obtained plasmid was transformed into E. coli DH5α (manufactured by Yeastern Biotech Co., Ltd.), and colony PCR was conducted with the transformed E. coli DH5α. The constructed plasmid was named pAC-psb (SEQ ID NO. 6).

Then, the plasmid pAC559em was used as a template and PCR was performed to amplify the DNA fragment of the erythromycin promoter with a pair of designed forward primer (EMP-F) and reverse primer (EMP-R) (SEQ ID NO. 9 and 10, respectively, manufactured by Tri-I Biotech, Inc.). The amplified DNA fragment of the erythromycin promoter was purified by PCR Purification kit (manufactured by Qiagen Inc.). The restriction enzyme reaction was performed on the plasmid pAC-psb and the DNA fragment of the erythromycin promoter with the restriction enzymes, SalI and NdeI (manufactured by New England Biolabs, USA). After the restriction enzyme reaction, the DNA fragment of the erythromycin promoter and pAC-psb were purified by Gel Extraction kit (Gel-M™ Gel Extraction System, manufactured by VIOGENE Co., Taiwan), and the ligation was performed on the DNA fragment of the erythromycin promoter and pAC-psb with a ratio of 3:1. The thus-obtained vector was transformed into E. coli DH5α, and colony PCR was conducted with the transformed E. coli DH5α. The constructed vector was named pAC-em (as shown in FIG. 2, deposited under BCRC Accession No. BCRC 940573 and DSMZ Accession No. DSM 22996).

### Example 2

### Construction of Expression Vector, pAC-em-gene of Protein

In the present invention, persons skilled in the art can use any known methods to insert a gene encoding a protein into the expression vector. For example, when pAC-em is used to express a gene encoding a protein in a Cyanobacterium, the gene encoding the protein is inserted into pAC-em by one or more restriction sites, preferably the restriction sites in pAC-em, or by any known means in the genetic engineering, such as Klenow fill-in followed by blunt end ligation, or adaptors.

### Example 3

### Construction of Expression Vector, pAC-em-EGFP

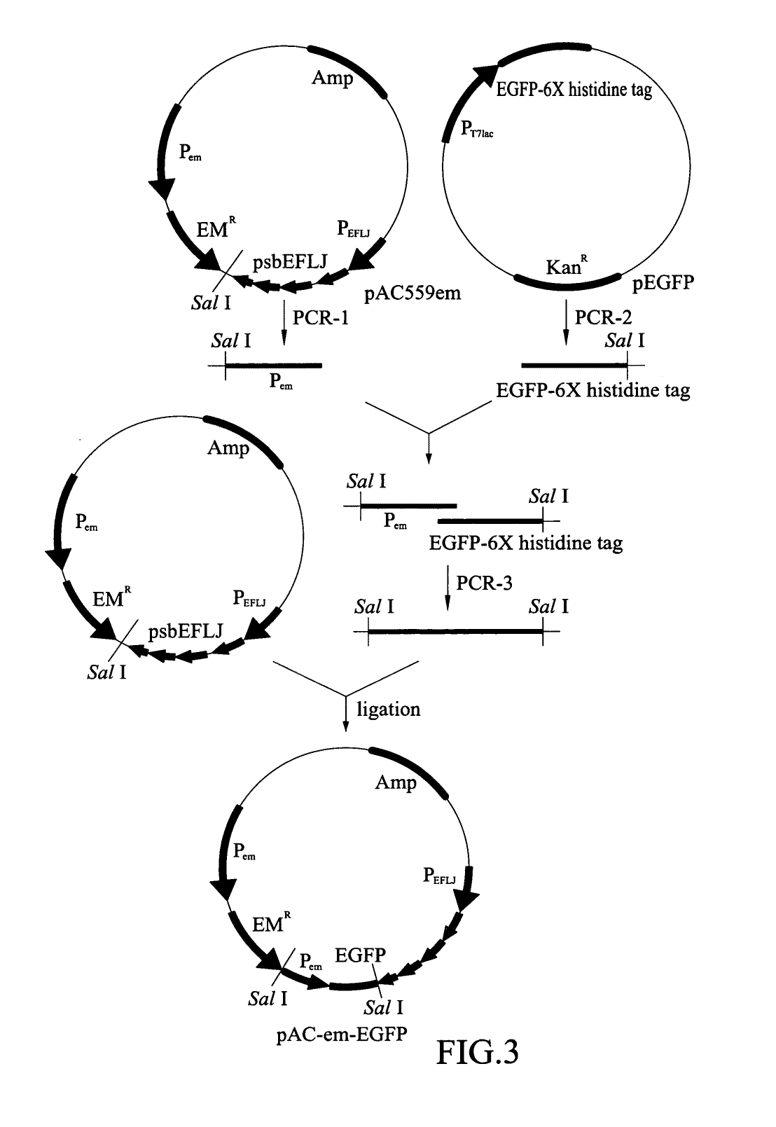

With respect to the construction of pAC-em-EGFP as shown in FIG. 3, pEGFP (manufactured by Clontech) and pAC559em were used as templates, and PCR was performed to amplify the EGFP-6× histidine tag gene and the erythromycin promoter (i.e. Pₑₘ) with designed forward primers and reverse primers (manufactured by Tri-I Biotech, Inc.). The amplified EGFP-6× histidine tag gene and the amplified Pₑₘ were purified by PCR Purification kit. The EGFP-6× histidine tag gene and the Pₑₘ were used as templates and PCR was performed again with a pair of designed primers (manufactured by Tri-I Biotech, Inc.) to obtain a Pₑₘ-EGFP-6× histidine tag gene fragment. The restriction enzyme reaction was performed on pAC559em and the Pₑₘ-EGFP-6× histidine tag gene fragment with the restriction enzyme, SalI. After the restriction enzyme reaction, the Pₑₘ-EGFP-6× histidine tag gene fragment and pAC559em were purified by Gel Extraction kit, and the ligation was performed on the Pₑₘ-EGFP-6× histidine tag gene fragment and pAC559em with a ratio of 3:1. The thus-obtained product was transformed into E. coli DH5α and colony PCR was conducted with the transformed E. coli DH5α. The constructed expression vector was named pAC-em-EGFP. Specific steps are described as follows.

### Synthesis of EGFP Gene and Restriction Enzyme Reaction

### First PCR

1. The following components were added to 0.2 mL PCT vials and sterile water was added to obtain the total volume of 100 μL and then homogenized.

PCR-1

| | | Final | |
|---|---|---|---|
| | Volume (μL) | Concentration | |
| Template (pAC559em) | 1 | 10-100 | ng |
| Forward primer (EM-F; | 1 | 0.1 | μM |
| SEQ ID NO. 13) | | | |
| Reverse primer (EM-EGFP-R; | 1 | 0.1 | μM |
| SEQ ID NO. 14) | | | |
| H₂O | 47 | | |

| | | |
|---|---|---|
| 2X GoTaq Green Master Mix | 50 | 1 X |
| (manufactured by Promega, USA) | | |

PCR-2

| | |
|---|---|
| | Final |
| Volume(μL) | Concentration |

| | | | |
|---|---|---|---|
| Template (pEGFP) | 1 | 10-100 | ng |
| Forward primer(EGFP-F(EM); | 1 | 0.1 | μM |
| SEQ ID NO. 16) | | | |
| Reverse primer (EGFP-R(EM); | 1 | 0.1 | μM |
| SEQ ID NO. 17) | | | |
| H₂O | 47 | | |

| | | |
|---|---|---|
| 2X GoTaq Green Master Mix | 50 | 1 X |

2. The vials were placed in the PCR machine for reaction and the condition for PCR is listed as follows.

| Hold 1 | 3 | min | 94° C. | |
|---|---|---|---|---|
| Cycle | 30 | sec | 94° C. | (Denaturation) |
| | 30 | sec | 55° C. | (Annealing) |
| | 1 | min | 72° C. | (Elongation) |
| | Cycle | | 30 | |
| Hold 2 | 7 | min | 72° C. | |

| | |
|---|---|
| ∞ | 4° C. |

3. After the above reaction, each PCR product (2 μL), 20×SYBR Green (2 μL) (manufactured by Molecular Probes, Inc, USA.), 6×DNA loading dye (1 μL) (manufactured by Protech Technology Enterprise Co., Ltd) and TE buffer (7 μL) (manufactured by Qiagen Inc.) were added to a vial (0.6 mL) and homogenized. The vial was stood in the dark for 10 minutes and subsequently, DNA electrophoresis was performed. The product (Pₑₘ) resulted from PCR-1 was 1033 bp and the product (EGFP-6× histidine tag gene) resulted from PCR-2 was 737 bp.
4. Remaining primers and enzyme were removed from the PCR products by PCR Purification kit (manufactured by Qiagen Inc.), such that the PCR products were purified for later use.

### Second PCR

1. The following components were added to a PCT vial (0.2 mL) and the sterile water was added to obtain the total volume of 100 μL and homogenized.

PCT-3

| | |
|---|---|
| | Final |
| Volume(μL) | Concentration |

| | | | |
|---|---|---|---|
| PCR-1:PCR-2 = 1:1 | 2 | 10-100 | ng |
| Forward primer (EM-F; | 1 | 0.1 | μM |
| SEQ ID NO. 13) | | | |
| Reverse primer (EGFP-R(EM); | 1 | 0.1 | μM |
| SEQ ID NO. 17) | | | |
| H₂O | 46 | | |

| | | |
|---|---|---|
| 2X GoTaq Green Master Mix | 50 | 1 X |

2. The vial was placed in the PCR machine and the condition for PCR is listed as follows.

| Hold 1 | 3 | min | 94° C. | |
|---|---|---|---|---|
| Cycle | 30 | sec | 94° C. | (Denaturation) |
| | 30 | sec | 55° C. | (Annealing) |
| | 90 | sec | 72° C. | (Elongation) |
| | Cycle | | 30 | |
| Hold 2 | 7 | min | 72° C. | |

| | |
|---|---|
| ∞ | 4° C. |

3. After the above reaction, the thus-obtained PCR product (2 μL), 20×SYBR Green (2 μL), 6×DNA loading dye (1 μL) and TE buffer (7 μL) were added to a vial (0.6 mL) and homogenized. The vial was stood in the dark for 10 minutes and subsequently, DNA electrophoresis was performed. The product, Pₑₘ-EGFP-6× histidine tag gene fragment, resulted from this PCR was 1770 bp.
4. Remaining primers and enzyme were removed from the PCR product by PCR Purification kit.
5. The restriction enzyme reaction was performed on the purified PCR product with the restriction enzyme, SalI. Amounts of components used in the restriction enzyme reaction are as follows.

| | | Final |
|---|---|---|
| | Volume(μL) | Concentration |
| Purified PCT Product | 30 | |
| BSA (100 X; manufactured by | 0.5 | 1 X |
| Sigma, St. Louis, MO., USA) | | |
| NEB Buffer 3 (10 X; manufactured | 5 | 1 X |
| by New England Biolabs, Inc.) | | |
| SalI | 1.5 | |
| H₂O | 13 | |

6. A mixture containing the above components was placed in a 37° C. water bath for 1 hour to carry out the restriction enzyme reaction.
7. DNA fragments were separated by DNA electrophoresis. The gel with the desired DNA fragment was cut and the desired DNA fragment was purified by Gel Extraction kit. The desired DNA fragment was measured by a RNA/DNA calculator (manufactured by Pharmacia) to determine the concentration and purity of the DNA fragment.
   Restriction Enzyme Reaction on pAC559em
1. The restriction enzyme reaction was performed on pAC559em with SalI. Amounts of components used in the restriction enzyme reaction are shown as follows.

| | | Final |
|---|---|---|
| | Volume(μL) | Concentration |
| pAC559em | 30 | |
| BSA (100 X) | 0.5 | 1 X |
| NEB Buffer 3 (10 X) | 5 | 1 X |
| SalI | 1.5 | |
| H₂O | 13 | |

2. A mixture containing the above components was placed in a 37° C. water bath for 1 hour to carry out the restriction enzyme reaction.
3. DNA fragments were separated by DNA electrophoresis. The gel with the desired DNA fragment was cut and the desired DNA fragment was purified by Gel Extraction kit. The desired DNA fragment was measured by a RNA/DNA calculator to determine the concentration and purity of the DNA fragment.

### Ligation

1. The Pₑₘ-EGFP-6× histidine tag gene fragment and pAC559em obtained from the above the restriction enzyme reactions and gel extractions were homogenized in a ratio of 3:1 and were added to a microtube (1.7 mL) with other components as shown in the following.

| | Volume(μL) |
|---|---|
| pAC559em | 5 |
| Pₑₘ-EGFP-6X histidine tag gene | 15 |
| fragment | |
| Ligase buffer (10 X; manufactured by | 2 |
| New England Biolabs, USA) | |
| PEG 4000 (manufactured by Sigma) | 2 |
| T4 DNA ligase (manufactured by New | 0.5 |
| England Biolabs, USA) | |
| H₂O | to 20 |

2. The microtube was placed in a 22° C. water bath for 1 hour to carry out ligation.
3. The ligated product was mixed with E. coli DH5α, stood for 30 minutes, and then heated for 30 seconds in a 42° C. water bath.
4. The above product was added to 1 mL LB (0.5% yeast extract (manufactured by Difco), 1% tryptone (manufactured by Difco), and 1% NaCl) in a laminar flow work station, and then incubated in an incubator (37° C., 170 rpm). After one hour incubation, the incubated product was applied to a LB agar plate with an antibiotic, Ampicillin (hereinafter abbreviated as Amp; manufactured by Sigma), to screen the E. coli DH5α.
   Colony PCR for pAC-em-EGFP
1. After the above screening, the growth colony was transferred to another agar plate with Amp by a sterile needle for replicating the colony. The agar plate was placed in a 37° C. incubator for 6 hours to allow the colony to grow.
2. The following components were premixed and an aliquot amount (20 μL) of the pre-mixture was added to a microtube (0.2 mL).

| | | Final | |
|---|---|---|---|
| | Volume(μL) | Concentration | |
| Template | — | — | |
| 10 μM Forward primer | 5 | 0.1 | μM |
| (pAC559em-F; | | | |
| SEQ ID NO. 11) | | | |
| 10 μM Reverse primer | 5 | 0.1 | μM |
| (pAC559em-R; | | | |
| SEQ ID NO. 12) | | | |
| H₂O | 240 | | |

| | | |
|---|---|---|
| 2X GoTaq Green Master Mix | 250 | 1 X |

3. The growth colony was added to the microtube by a sterile needle. The microtube was placed in the PCR machine and the condition for PCR is as follows.

| Hold 1 | 3 | min | 94° C. | |
|---|---|---|---|---|
| Cycle | 30 | sec | 94° C. | (Denaturation) |
| | 30 | sec | 55° C. | (Annealing) |
| | 70 | sec | 72° C. | (Elongation) |
| | Cycle | | 30 | |
| Hold 2 | 7 | min | 72° C. | |

| | |
|---|---|
| ∞ | 4° C. |

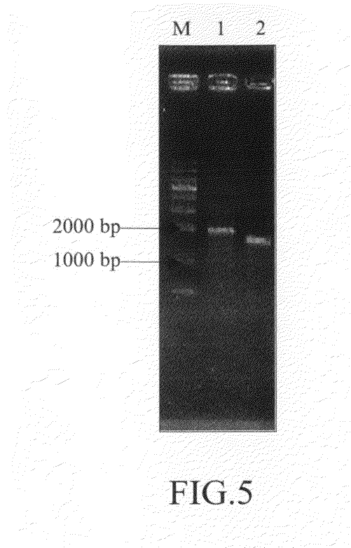

4. The thus-obtained PCR product (2 μL), 20×SYBR Green (2 μL), 6×DNA loading dye (1 μL) and TE buffer (7 μL) were added to a vial (0.6 mL) and homogenized. The vial was stood in the dark for 10 minutes and subsequently, DNA electrophoresis was performed.
5. After the DNA from the colony PCR positive colony was sequenced and confirmed (as shown in FIG. 5), the expression vector was purified by Plasmid DNA Extraction System (Mini-M™, manufactured by VIOGENE Co., Taiwan) and was transformed into Synechocystis sp. PCC 6803 for expression.

### Example 4

### Construction of Expression Vector, pAC-em-VHb

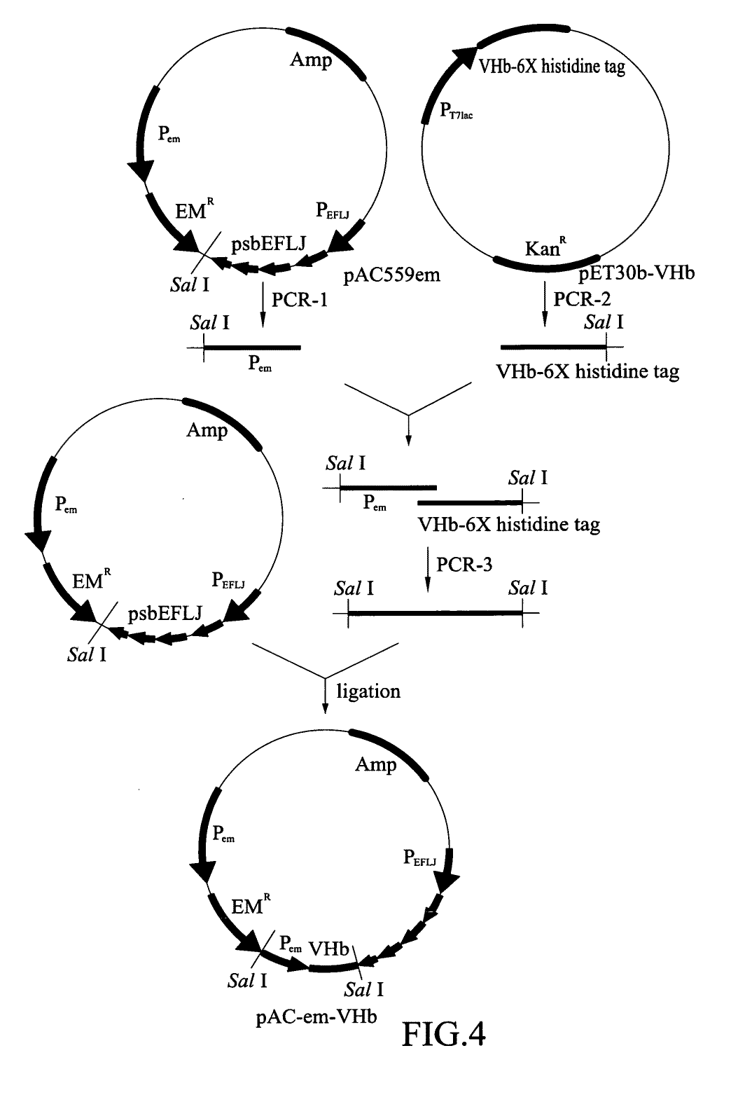

With respect to the construction of pAC-em-VHb as shown in FIG. 4, pAC559em and pET30b-VHb (SEQ ID NO. 7) were used as templates, and PCR was performed to amplify the erythromycin promoter (i.e. Pₑₘ) and the VHb-6× histidine tag gene with designed forward primers and reverse primers (manufactured by Tri-I Biotech, Inc.). The amplified VHb-6× histidine tag gene and the amplified Pₑₘ were purified by PCR Purification kit (manufactured by Qiagen Inc.). The VHb-6× histidine tag gene and Pₑₘ were used as templates and PCR was performed again with a pair of designed primers (manufactured by Tri-I Biotech, Inc.) to obtain a Pₑₘ-VHb-6× histidine tag gene fragment. The restriction enzyme reaction was performed on pAC559em and the Pₑₘ-VHb-6× histidine tag gene fragment with the restriction enzyme, SalI (manufactured by New England Biolabs, USA). After the restriction enzyme reaction, the Pₑₘ-VHb-6× histidine tag gene fragment and pAC559em were purified by Gel Extraction kit, and ligation was performed on the Pₑₘ-VHb-6× histidine tag gene fragment and pAC559em with a ratio of 3:1. The thus-obtained product was transformed into E. coli DH5α and colony PCR was conducted with the transformed E. coli DH5α. The constructed expression vector was named pAC-em-VHb. Specific steps are described as follows.

### Synthesis of VHb Gene and Restriction Enzyme Reaction

### First PCR

1. The following components were added to PCT vials (0.2 mL) and sterile water was added to obtain the total volume of 100 μL and then homogenized.

PCR-1

| | | Final | |
|---|---|---|---|
| | Volume (μL) | Concentration | |
| Template (pAC559em) | 1 | 10-100 | ng |
| Forward primer | 1 | 0.1 | μM |
| (EM-F; | | | |
| SEQ ID NO. 13) | | | |
| Reverse primer | 1 | 0.1 | μM |
| (EM-VHb-R; | | | |
| SEQ ID NO. 15) | | | |
| H₂O | 47 | | |

| | | |
|---|---|---|
| 2X GoTaq Green Master Mix | 50 | 1 X |

PCR-2

| | | Final | |
|---|---|---|---|
| | Volume(μL) | Concentration | |
| Template (pET30b-VHb) | 1 | 10-100 | ng |
| Forward primer | 1 | 0.1 | μM |
| (VHb-F(EM); | | | |
| SEQ ID NO. 18) | | | |
| Reverse primer | 1 | 0.1 | μM |
| (VHb-R(EM); | | | |
| SEQ ID NO. 19) | | | |
| H₂O | 47 | | |

| | | |
|---|---|---|
| 2X GoTaq Green Master Mix | 50 | 1 X |

2. The vials were placed in the PCR machine and the condition for PCR is as follows.

| Hold 1 | 3 | min | 94° C. | |
|---|---|---|---|---|
| Cycle | 30 | sec | 94° C. | (Denaturation) |
| | 30 | sec | 55° C. | (Annealing) |
| | 1 | min | 72° C. | (Elongation) |
| | Cycle | | 30 | |
| Hold 2 | 7 | min | 72° C. | |

| | |
|---|---|
| ∞ | 4° C. |

3. After the above reaction, each PCR product (2 μL), 20×SYBR Green (2 μL), 6×DNA loading dye (1 μL) and TE buffer (7 μL) were added to a vial (0.6 mL) and homogenized. The vial was stood in the dark for 10 minutes and subsequently, DNA electrophoresis was performed. The product (Pₑₘ) resulted from PCR-1 was 1033 bp, and the product (VHb-6× histidine tag gene) resulted from PCR-2 was 437 bp.
4. Remaining primers and enzyme were removed from the PCR products by PCR Purification kit (manufactured by Qiagen Inc.), such that the PCR products were purified for later use.

### Second PCR

1. The following components were added to a PCT vial (0.2 mL) and the sterile water was added to obtain the total volume of 100 μL and homogenized.

PCT-3

| | | Final | |
|---|---|---|---|
| | Volume(μL) | Concentration | |
| PCR-1:PCR-2 = 1:1 | 2 | 10-100 | ng |
| Forward primer | 1 | 0.1 | μM |
| (EM-F; | | | |
| SEQ ID NO. 13) | | | |
| Reverse primer | 1 | 0.1 | μM |
| (VHb-R(EM); | | | |
| SEQ ID NO. 19) | | | |
| H₂O | 46 | | |

| | | |
|---|---|---|
| 2X GoTaq Green Master Mix | 50 | 1 X |

2. The vial was placed in the PCR machine and the condition for PCR is listed as follows.

| Hold 1 | 3 | min | 94° C. | |
|---|---|---|---|---|
| Cycle | 30 | sec | 94° C. | (Denaturation) |
| | 30 | sec | 55° C. | (Annealing) |
| | 90 | sec | 72° C. | (Elongation) |
| | Cycle | | 30 | |
| Hold 2 | 7 | min | 72° C. | |

| | |
|---|---|
| ∞ | 4° C. |

3. After the above reaction, the thus-obtained PCR product (2 μL), 20×SYBR Green (2 μL), 6×DNA loading dye (1 μL) and TE buffer (7 μL) were added to a vial (0.6 mL) and homogenized. The vial was stood in the dark for 10 minutes and subsequently, DNA electrophoresis was performed. The product, Pₑₘ-VHb-6× histidine tag gene fragment, resulted from this PCR was 1470 bp.
4. Remaining primers and enzyme were removed from the PCR product by PCR Purification kit.
5. The restriction enzyme reaction was performed on the purified PCR product with the restriction enzyme, SalI. Amounts of components used in the restriction enzyme reaction are as follows.

| | | Final |
|---|---|---|
| | Volume(μL) | Concentration |
| Purified PCT Product | 30 | |
| BSA (100 X) | 0.5 | 1 X |
| NEB Buffer 3 (10 X) | 5 | 1 X |
| SalI | 1.5 | |
| H₂O | 13 | |

6. A mixture containing the above components was placed in a 37° C. water bath for 1 hour to carry out the restriction enzyme reaction.
7. DNA fragments were separated by DNA electrophoresis. The gel with the desired DNA fragment was cut and the desired DNA fragment was purified by Gel Extraction kit. The desired DNA fragment was measured by a RNA/DNA calculator to determine the concentration and purity of the DNA fragment.
   Restriction Enzyme Reaction on pAC559em
1. The restriction enzyme reaction was performed on pAC559em with SalI. Amounts of components used in the restriction enzyme reaction are shown as follows.

| | | Final |
|---|---|---|
| | Volume(μL) | Concentration |
| pAC559em | 30 | |
| BSA (100 X) | 0.5 | 1 X |
| NEB Buffer 3 (10 X) | 5 | 1 X |
| SalI | 1.5 | |
| H₂O | 13 | |

2. A mixture containing the above components was placed in a 37° C. water bath for 1 hour to carry out the restriction enzyme reaction.
3. DNA fragments were separated by DNA electrophoresis. The gel with the desired DNA fragment was cut and the desired DNA fragment was purified by Gel Extraction kit. The desired DNA fragment was measured by a RNA/DNA calculator to determine the concentration and purity of the DNA fragment.

### Ligation

1. The Pₑₘ-VHb-6× histidine tag gene fragment and pAC559em obtained from the above the restriction enzyme reactions and gel extractions were homogenized in a ratio of 3:1 and were added to a microtube (1.7 mL) with other components as shown in the following.

| | Volume (μL) |
|---|---|
| pAC559em | 5 |
| Pₑₘ-VHb-6X histidine tag gene | 15 |
| fragment | |
| Ligase buffer (10 X) | 2 |
| PEG 4000 | 2 |
| T4 DNA ligase | 0.5 |
| H₂O | to 20 |

2. The microtube was placed in a 22° C. water bath for 1 hour to carry out ligation.
3. The ligated product was mixed with E. coli DH5α, stood for 30 minutes, and then heated for 30 seconds in a 42° C. water bath.
4. The above product was added to 1 mL LB (0.5% yeast extract, 1% tryptone and 1% NaCl) in a laminar flow work station, and then incubated in an incubator (37° C., 170 rpm). After one hour incubation, the incubated product was applied to a LB agar plate with Amp to screen the E. coli DH5α.
   Colony PCR for pAC-em-EGFP
1. After the above screening, the growth colony was transferred to another agar plate with Amp by a sterile needle for replicating the colony. The agar plate was placed in a 37° C. incubator for 6 hours to allow the colony to grow.
2. The following components were premixed and an aliquot amount (20 μL) of the pre-mixture was added to a microtube (0.2 mL).

| | | Final | |
|---|---|---|---|
| | Volume(μL) | Concentration | |
| Template | — | — | |
| 10 μM Forward primer | 5 | 0.1 | μM |
| (pAC559em-F; | | | |
| SEQ ID NO. 11) | | | |
| 10 μM Reverse primer | 5 | 0.1 | μM |
| (pAC559em-R; | | | |
| SEQ ID NO. 12) | | | |
| H₂O | 240 | | |

| | | |
|---|---|---|
| 2X GoTaq Green Master Mix | 250 | 1 X |

3. The growth colony was added to the microtube by a sterile needle. The microtube was placed in the PCR machine and the condition for PCR is as follows.

| Hold 1 | 3 | min | 94° C. | |
|---|---|---|---|---|
| Cycle | 30 | sec | 94° C. | (Denaturation) |
| | 30 | sec | 55° C. | (Annealing) |
| | 70 | sec | 72° C. | (Elongation) |
| | Cycle | | 30 | |
| Hold 2 | 7 | min | 72° C. | |

| | |
|---|---|
| ∞ | 4° C. |

4. The thus-obtained PCR product (2 μL), 20×SYBR Green (2 μL), 6×DNA loading dye (1 μL) and TE buffer (7 μL) were added to a vial (0.6 mL) and homogenized. The vial was stood in the dark for 10 minutes and subsequently, DNA electrophoresis was performed.
5. After the DNA from the colony PCR positive colony was sequenced and confirmed (as shown in FIG. 5), the expression vector was purified by Plasmid DNA Extraction System and was transformed into Synechocystis sp. PCC 6803 for expression.

### Example 5

### Culture and Screening of Synechocystis sp. PCC 6803

1. Monoclonal Synechocystis sp. PCC 6803 was added to a 4 mL BG-11 medium and incubated for about 4 days at 30° C. and 200 rpm. The medium with Synechocystis sp. PCC 6803 was diluted 16 times in a 50 mL BG-11 medium and incubated at 30° C. and 200 rpm until A₇₃₀ was about 0.4 (measured by UV/VIS Spectrophotometer V-530 manufactured by Model Jasco).

BG-11 medium

| COMPONENT | (g/L) | COMPONENT | (g/L) |
|---|---|---|---|
| H₃BO₃ | 2.86 × 10−3 | NaNO₃ | 1.496 |
| MnCl₂•4H₂O | 1.81 × 10−3 | Citric acid | 0.006 |
| ZnSO₄•7 H₂O | 0.22 × 10−3 | diNaEDTA | 0.001 |
| Na₂MoO₄•2 H₂O | 0.39 × 10−3 | K₂HPO₄ | 0.03 |
| CuSO₄•5 H₂O | 7.90 × 10−5 | Na₂CO₃ | 0.02 |
| Co(NO₃)₂•6 H₂O | 4.94 × 10−5 | Ferric ammonium | 0.006 |
| | | citrate | |
| MgSO₄•7 H₂O | 0.075 | TES | 1.255 |
| CaCl₂•2 H₂O | 0.036 | | |

TES: N-Tris(hydroxylmethyl)methyl-2-aminoethanesulfonic acid (C₆H₁₃NO₆S₂)

2. 40 μL of the thus-obtained medium was centrifuged (3400 rpm, 15 min).
3. Supernatant was removed and a fresh BG-11 medium was added, so that A₇₃₀ was 2.5 (measured by UV/VIS Spectrophotometer).
4. 300 μL of the medium with Synechocystis sp. PCC 6803 was added to a 1.5 mL tube.
5. 6 μL of the obtained expression vector was added to the tube and homogenized.
6. The tube was placed in a 30° C. incubator for 4 hours and gently shaken every 30 minutes.
7. A BG-11 agar plate with glucose (manufactured by Acros, USA) was prepared.
8. A sterile filter (0.22 μm) was placed on the agar plate.
9. 100 μL of the medium with Synechocystis sp. PCC 6803 containing the expression vector was evenly spread on the filter.
10. The filter was incubated in a 30° C. incubator for 24 hours.
11. A BG-11 agar plate with an antibiotic, erythromycin (manufactured by Sigma), was prepared.
12. The filter with colonies was transferred to the BG-11 agar plate with erythromycin.
13. The agar plate was incubated in a 30° C. incubator for several days until green colonies grew.

### Example 6

### Extraction of Genomic DNA of Synechocystis sp. PCC 6803

1. Monoclonal Synechocystis sp. PCC 6803 was added to a 3 mL BG-11 medium until log growth phase (about 7 days). Then, the medium was centrifuged at 7500 rpm for 10 min.
2. Supernatant was removed and Blood & Tissue Genomic DNA Extraction System (manufactured by Viogene-BioTek Corporation) was used to extract genomic DNA. 200 μL of the lysozyme reaction solution (20 mM Tris-HCl, pH 8.0, 2 mM EDTA, 20 mg/mL lysozyme) was added and incubated at 37° C. for 30 minutes.
3. 20 μL Protease K and 200 μL EX buffer were added and the mixture was vortexed immediately for 20 seconds.
4. The mixture was incubated at 60° C. for 30 minutes and vortexed once every 5 minutes.
5. The mixture was incubated at 70° C. for 30 minutes.
6. ddH₂O was pre-heated at 70° C.
7. 210 μL isopropanol (manufactured by Acros organics N.V./S.A.) was added to the mixture.
8. The suspension obtained from the step 7 was put in a B/T Genomic DNA Column and centrifuged at 8000 rpm for 2 min. Then, the filtered liquid was removed from the B/T Genomic DNA Column.
9. 0.5 mL WS buffer was added to the B/T Genomic DNA Column and the B/T Genomic DNA Column was centrifuged at 8000 rpm for 2 min. The filtered buffer was removed from the B/T Genomic DNA Column. This step was repeated again.
10. The B/T Genomic DNA Column was centrifuged at a high speed (˜12000 rpm) to remove the remaining buffer.
11. The B/T Genomic DNA Column was transferred to a new 1.5 mL microtube and 50 μL of the pre-heated ddH₂O was added to the column.
12. The column was stood for 5 minutes and centrifuged at a high speed (˜12000 rpm) for 2 minutes to obtain the genomic DNA.

### Example 7

### Colony Culture and Production of Recombinant Protein

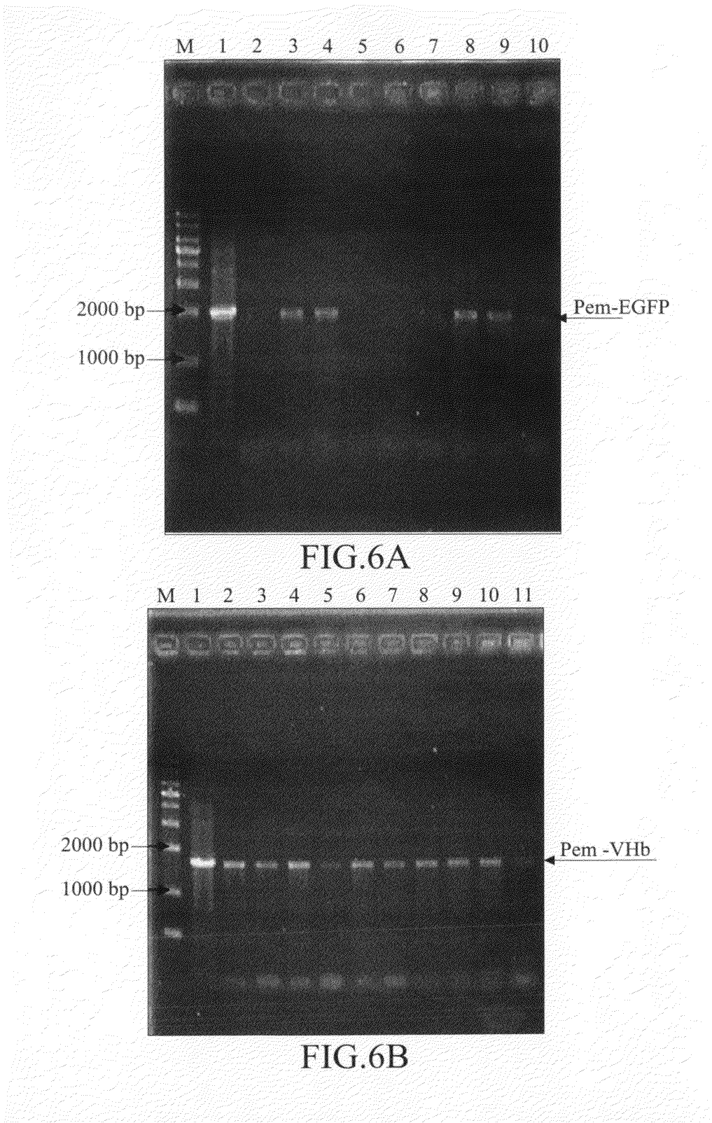

The constructed expression vectors entered Synechocystis sp. PCC 6803 by natural transformation and homologous recombination was performed at the cytochrome b559 gene of the chromosome. It is known from references that 4-5 hours are the best for transformation (Galyna I. Kufryk, Monika Sachet, Georg Schmetterer, Wim F. J. Vermaas, FEMS Microbiology Letters, 206 (2002) 215-219; Xiaonan Zang, Bin Liu, Shunmei Liu, K. K. I. U. Arunakumara, and Xuecheng Zhang, The Journal of Microbiology, Vol. 45: p. 241-245, 2007). Not all of single colonies contained the foreign genes. Therefore, those colonies were transferred to another BG-11 agar plate with erythromycin for screening. After four screenings, the genetically modified Synechocystis sp. PCC 6803 was obtained and its genomic DNA was confirmed by colony PCR. FIG. 6A shows the screening results for pAC-em-EGFP, in which six out of night colonies contained recombinant DNA from pAC-em-EGFP. FIG. 6B shows the screening results for pAC-em-VHb, in which 10 colonies contained the recombinant DNA from pAC-em-VHb.

Synechocystis sp. PCC 6803 was suspended in 2.5 mL PBS buffer and placed on ice. Synechocystis sp. PCC 6803 was disrupted by ultrasonic processor (manufactured by Microson). The disruption was performed and stopped every 10 seconds and Synechocystis sp. PCC 6803 was ice bathed for 20 seconds. The above disruption was repeated 15 times. During this disruption, Synechocystis sp. PCC 6803 was placed on ice. The thus-obtained solution was separated by centrifugation at 4° C. and 12000 rpm for 10 min. The resulted supernatant full of the soluble protein was removed. The resulted pellet was washed by PBS buffer and was dissolved in 2.5 mL 8 M urea (manufactured by Acros organics N.V./S.A) to obtain the insoluble protein.

### Example 8

### Bradford Method for Protein Analysis

1. BSA (manufactured by Sigma, St. Louis, Mo., USA) with a standard concentration of 1440 μg/mL was diluted by a 0.15 N NaCl solution (manufactured by Acros organics N.V./S.A.) to obtain solutions with concentrations of from 10 to 144 μg/mL BSA. The BSA solutions were independently distributed to 1.5 mL microtubes.
2. 1 mL Coomassie brilliant solution (Coomassie Brilliant Blue R-250; manufactured by Amresco, Solon, Ohio, USA) was added to the diluted BSA solutions (100 μL for each concentration), and then homogenized. The reaction was performed at room temperature for 10 minutes. An OD₅₉₅ value of each diluted BSA solution was measured.
3. A standard curve was established through OD₅₉₅ values and the concentrations of the diluted BSA solutions.
4. 10 μL of a sample with an unknown concentration obtained from Synechocystis sp. PCC 6803 having the recombinant DNA was diluted by 0.15 N NaCl to obtain 100 μL, and 1 mL Coomassie brilliant solution was added and homogenized for reaction at room temperature for 10 minutes.
5. An OD₅₉₅ value was measured by UV/VIS Spectrophotometer (manufactured by Model Jasco V-530) and the concentration for the sample would be obtained according the standard curve.

### Example 9

### Protein Electrophoresis

According to Example 7, the amount of Synechocystis sp. PCC 6803 containing the expression vector was determined when the amount of the protein was 50 μg. A pre-cast polyacrylamide gel (manufactured by ProTech) and electrophoresis buffer were prepared, and then the electrophoresis buffer was poured into a gel holder to cover the gel. 4× loading dye (manufactured by ProTech) was mixed with the protein, and both were heated at 95° C. for 10 min. After the protein was cooled to room temperature, 10 μL of the protein was loaded into a lane. 1× running buffer (manufactured by ProTech) was added to buffer chambers of an electrophoresis apparatus. Electric wires were connected to a power supply (Power Pac200, manufactured by BIO-RAD), and the protein was driven until the front of the loading dye traveled to the bottom of the gel (110 V, for about 90 min). After the electrophoresis, the gel was separated from the glass plates and was submerged in a Coomassie Blue stain solution (0.2 Brilliant blue R (manufactured by Acros), 20 acetic acid and 50 methanol (manufactured by J. T. Baker)) with gentle shaking on a shaker for at least 30 min. The gel was removed from the solution and was placed in Destain buffer (10 acetic acid (manufactured by J. T. Baker) and 20 methanol (manufactured by J. T. Baker)) for 15 min to 1 hour.

### Example 10

### Western Blot

The above-obtained gel was placed on a filter paper, which was pre-wetted by CAPS buffer (3.3 g 3-cyclohexylamino-1-propanesulfonic acid and DI water until the total volume of 1.5 L, pH=11) and was on a transfer cassette, and then a PVDF membrane (as big as the gel in size) pre-wetted by the CAPS buffer was placed on the gel. Bubbles were removed by a 5 mL pipette. Another wet filter paper was placed on the top of the PVDF membrane. The cassette was closed and placed in a transfer apparatus with 24 volt for transfer. After the transfer, the PVDF membrane was placed in blocking buffer (TBST buffer (4.84 g Tris-HCl, 158 g NaCl pH=7.5, 21 mL Tween-20 and DI water to obtain the total volume of 2 L and pH=7) and skim milk powder) for 1.5 h or at 4° C. overnight. The PVDF membrane was washed for 10 min by the TBST buffer under slow shaking. This step was repeated four times. The PVDF membrane was placed in a plastic bag containing anti-polyhistidine antibody (1/5000 dilution with the blocking buffer (2.5 g skim milk powder (5) and 50 mL TBST buffer) for 1 h. Then, the PVDF membrane was washed by the TBST buffer for 10 min. This step was repeated four times. The PVDF membrane was placed in a plastic bag containing anti-mouse IgG alkalinephosphatase-conjugated antibody (1/5000 dilution with the blocking buffer) for 1 h. The PVDF membrane was washed by the TBST buffer for 10 min. This step was repeated four times. The PVDF membrane was reacted with a NBT/BCIP mixture (manufactured by PerkinElmer; 100 μL NBT/BCIP and 10 mL alkaline assay buffer (1.21 g Tris-HCl, 0.58 g NaCl, 0.1 g MgCl₂. 6H₂O and DI water to obtain a total volume of 100 mL and pH=9.5)) in a dark room until the color was clearly visualized on the PVDF membrane (about 10 min). TE buffer was added to stop the reaction.

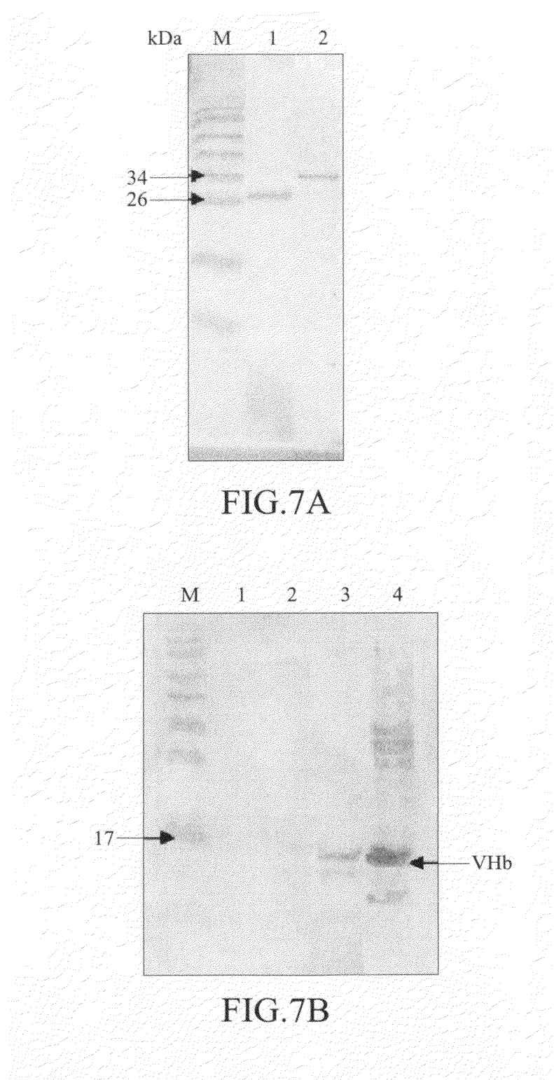

The PVDF membranes obtained from the above method are shown in FIGS. 7A and 7B. In FIG. 7A, [M] represents protein marker (Prestained Protein Ladder; manufactured by MBI Fermentas); [1] represents Synechocystis sp. PCC 6803/pAC-em-EGFP; and [2] represents BL21/pEGFP. In FIG. 7B, [M] represents protein marker (Prestained Protein Ladder); [1] represents Synechocystis sp. PCC 6803; [2] represents Synechocystis sp. PCC 6803/pAC559em; [3] represents Synechocystis sp. PCC 6803/pAC-em-VHb; and [4] represents E. coli DH5α/pAC-em-VHb.

As shown in the above results, the erythromycin promoter excellently drove the expressions of genes encoding different proteins in the Cyanobacteria.

### Example 11

### Erythromycin Induction

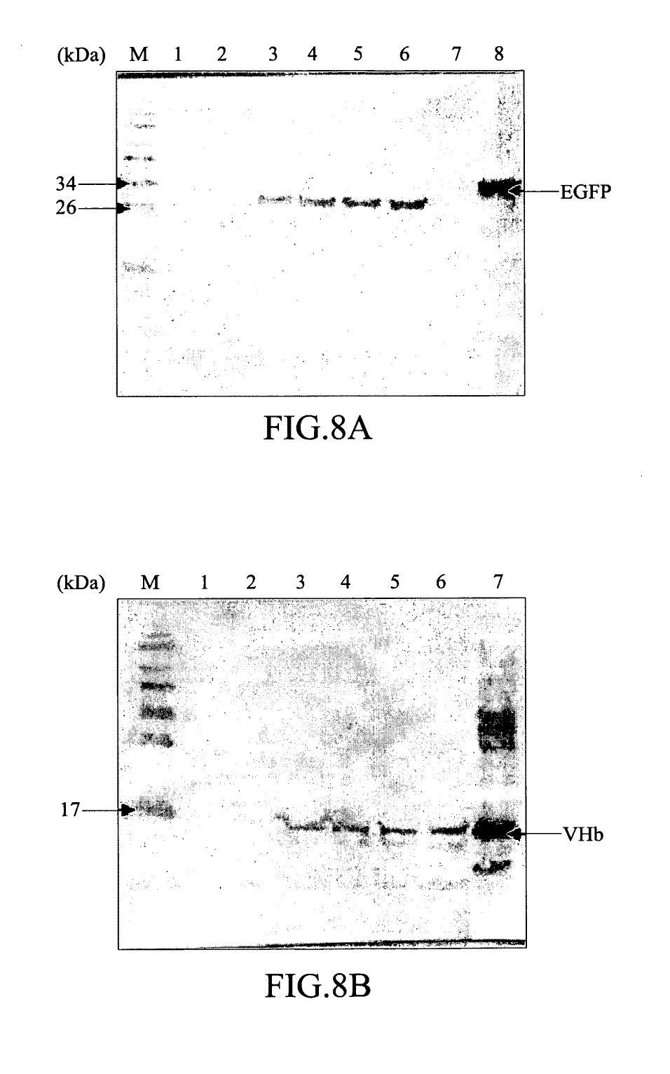

It is known that the erythromycin promoter is a constitutive promoter (Sucharu Horinouchi and Bernard Weisblum, Proceedings of the National Academy of Sciences of the USA, Vol. 77, 7079-7083, 1980). Thus, pAC-em-EGFP and pAC-em-VHb could express EGFP and VHb, respectively, without the induction of erythromycin. However, the expressions of EGFP and VHb in Synechocystis sp. PCC 6803 could also be induced by the erythromycin with different concentrations. Synechocystis sp. PCC 6803 with pAC-em-EGFP or pAC-em-VHb was applied to a BG-11 plate with 5 mM glucose and 0.1 μg/mL of the erythromycin for 2 days (OD₇₃₀ was about 2). The erythromycin (manufactured by Sigma, St. Louis, Mo., USA) with different concentrations was added to the plates and the plates were incubated for about one day. Western blot was performed with 1 mL of Synechocystis sp. PCC 6803 having pAC-em-EGFP or pAC-em-VHb. As shown in FIGS. 8A and 8B, when the erythromycin concentration was increased, the protein expression of EGFP or VHb was getting higher. Therefore, the amount of the protein expressed in Synechocystis sp. PCC 6803 could be increased by the erythromycin if need be.

The foregoing descriptions of the detailed embodiments are only illustrated to disclose the principle and functions of the present invention and do not restrict the scope of the present invention. It should be understood to those in the art that all modifications and variations according to the spirit and principle in the disclosure of the present invention should fall within the scope of the appended claims. It is intended that the specification and examples are considered as exemplary only, with a true scope of the invention being indicated by the following claims.

What is claimed is:

1. An expression vector comprising a protein expression cassette and a homologous recombination DNA fragment flanking the protein expression cassette, wherein the protein expression cassette comprises an erythromycin promoter.

2. The expression vector of claim 1, wherein the homologous recombination DNA fragment comprises a psbE gene, a psbF gene, a psbL gene, a psbJ gene, a DNA fragment thereof or a combination thereof.

3. The expression vector of claim 1, wherein the protein expression cassette further comprises at least one restriction site.

4. The expression vector of claim 3, wherein the restriction site comprises AatI, ApaI, AscI, AspI, AvaI, BamHI, BglII, EagI, EcoNI, KpnI, MfeI, NspV, PacI, MunI, NspI, PmeI, PmlI, SfuI, SfiI, StuI, SwaI, NdeI, SalI, SpeI, XboI, XhoI, XmaII or a combination thereof.

5. The expression vector of claim 3, wherein the protein expression cassette comprises a gene encoding a protein that is inserted into the restriction site.

6. The expression vector of claim 5, wherein the gene encoding a protein comprises a gene encoding EGFP, a gene encoding VHb, a gene encoding a subunit of acetyl-CoA carboxylase, a gene encoding lysophosphatidic acid acyltransferase, a gene encoding chloroplast membrane-associated protein, a gene encoding 1-aminocyclopropane-1-carboxylic acid oxidase, a gene encoding 1-aminocyclopropane-1-carboxylic acid synthase, a gene encoding cis-aconitate decarboxylase, a gene encoding alcohol dehydrogenase, a gene encoding pyruvate decarboxylase or a combination thereof.

7. The expression vector of claim 1, wherein the protein expression cassette further comprises a gene encoding a tag.

8. The expression vector of claim 7, wherein the gene encoding the tag comprises a gene encoding flag 3 tag, a gene encoding 6× histidine tag or a combination thereof.

9. The expression vector of claim 1, which is deposited under DSMZ Accession No. DSM 22996.

10. The expression vector of claim 1, wherein the expression vector expresses in a Cyanobacterium.

11. The expression vector of claim 10, wherein the Cyanobacterium comprises Synechocystis sp., Synechococcus spp., Microcystis aeruginosa, Prochlorococcus marinus or Nostoc punctiforme.

12. A transformed Escherichia coli comprising the expression vector of claim 1.

13. A transformed Escherichia coli comprising the expression vector of claim 9.

14. A genetically modified Cyanobacterium comprising the expression vector of claim 1.

15. The genetically modified Cyanobacterium of claim 14, wherein the Cyanobacterium comprises Synechocystis sp., Synechococcus spp., Microcystis aeruginosa, Prochlorococcus marinus or Nostoc punctiforme.

16. A genetically modified Cyanobacterium comprising the expression vector of claim 9.

17. A method for expressing a protein in a Cyanobacterium, comprising:
transforming the expression vector of claim 1 into the Cyanobacterium.

18. The method of claim 17, wherein the Cyanobacterium comprises Synechocystis sp., Synechococcus spp., Microcystis aeruginosa, Prochlorococcus marinus or Nostoc punctiforme.

19. A use of the expression vector of claim 1 in expressing a protein.

20. A use of the genetically modified Cyanobacterium of claim 14 in expressing a protein.
